(19) United States
(12) Patent Application Publication
(10) Pub. No.: US 2025/0324963 A1
(43) Pub. Date: Oct. 23, 2025

(54) Treatment Systems and Associated Methods

(71) Applicant: Lisi Global, Inc., Richland, WA (US)

(72) Inventors: Jason D. Crisp, Newark, DE (US); Gordon J. McComb, Kennewick, WA (US)

(73) Assignee: Lisi Global, Inc., Richland, WA (US)

(21) Appl. No.: 18/994,001

(22) PCT Filed: Jul. 21, 2023
(86) PCT No.: PCT/US2023/028417

§ 371 Date: Jan. 13, 2025

Related U.S. Application Data
(60) Provisional application No. 63/391,586, filed on Jul. 22, 2022.

(51) Int. Cl. A01M 17/00

(52) U.S. Cl. CPC A01M 17/00

(57) ABSTRACT

Treatment systems and associated methods are described. According to one aspect, a treatment system includes a discharge assembly coupled with and configured to receive electrical energy from an input power source and to generate a plurality of pulses of electrical energy; a plurality of electrodes coupled with the discharge assembly, and wherein the electrodes are configured to apply the pulses of electrical energy to ground of a treatment location to manage pests within the ground of the treatment location; and wherein the pulses of electrical energy that are applied to the ground of the treatment location are a plurality of square waveform pulses.

### RELATED PATENT DATA

This application claims the benefit of U.S. Provisional Patent Application Ser. No. 63/391,586, filed Jul. 22, 2022, titled “Discharge Modules and Associated Methods,” the disclosure of which is incorporated herein by reference.

### TECHNICAL FIELD

This disclosure relates to treatment systems and associated methods.

### BACKGROUND OF THE DISCLOSURE

Organisms that are harmful to plants, crops, grass sporting surfaces, etc. occur naturally in the soil. Example harmful organisms include bacteria, viruses, fungus, worms, and insects. More specific examples of harmful organisms include the phylum nematoda (round worms) and different fungi including molds, yeasts, and mushrooms that typically cause the most severe crop losses in the world.

The use of resistant plant cultivars, and the eradication of fungi through the use of assorted cultural practices are some of the more well-known approaches which have been employed to address the diseases caused by various fungal pathogens. However, in many situations these well-known measures cannot be employed.

There are many different types and chemical classes of fungicides currently available. The current literature reports that fumigants, sometimes in conjunction with other chemical mitigants, have been the traditional means for controlling fungal plant pathogens and other plant pathogens and pests. Currently, fumigants are still used to control fungal pathogens in many countries, including the United States. However, the high cost of the available fumigants has restricted their use to high value crops in countries where these admittedly toxic products can be applied safely and effectively. Many countries have as of late severely restricted the use of fumigants, or completely banned them altogether as they have been recognized as a health and environmental hazard.

Some embodiments of the present disclosure described herein are directed towards commercially viable and environmentally friendly systems, apparatus and associated methods of controlling harmful organisms in-situ at a treatment location, and that are viable alternatives to conventional control apparatus and methods in agriculture.

### BRIEF DESCRIPTION OF THE DRAWINGS

Example embodiments of the disclosure are described below with reference to the following accompanying drawings.

FIG. 1 is a perspective view of a treatment system according to one embodiment.

FIG. 2 is a perspective view of electrodes and plants in a treatment location according to one embodiment.

FIG. 3 is an illustrative representation of square waveform pulses according to one embodiment.

FIG. 4 is a block diagram of a discharge assembly of a treatment system according to one embodiment.

FIG. 5 is a map showing how to assemble FIGS. 5A-5F.

FIGS. 5A-5F are schematic diagrams of one embodiment of circuitry of a discharge assembly once assembled as shown in FIG. 5.

FIG. 6A is a graphical representation of capacitive-decay pulses according to one embodiment.

FIG. 6B is a graphical representation of square waveform pulses according to one embodiment.

FIG. 7 is a graphical representation of waveforms of pulses generated by a discharge assembly of a treatment system according to one embodiment.

FIG. 8A is a perspective view of an underside of an electrode assembly according to one embodiment.

FIG. 8B is a perspective view of a top side of an electrode assembly according to one embodiment.

### DETAILED DESCRIPTION OF THE DISCLOSURE

Example organism treatment systems described herein include treatment systems configured to efficiently and effectively apply electrical energy from a discharge assembly to a treatment location. Example treatment locations include any volume, type, or condition of air, soil, water, and/or growing media, planted or fallow field and where a tree, vine, grass, weed, sports grass turf (e.g., golf turf), annual or perennial plant, or commodity may be present, or any type or condition of planting suitable for reception of electrical energy from the treatment system. The treatment systems and associated methods described herein may be utilized in many areas of horticulture as well as for treatment of commodities such as seeds, seedlings, saplings, starts, or plugs.

In some embodiments described herein, plural electrodes of the treatment system engage the treatment location and deliver electrical energy to a target volume of soil and other matter between the electrodes to control harmful organisms or otherwise provide a desired outcome at the treatment location that results in a reduction of the harmful organisms and increases plant vigor and growth. In some embodiments, the treatment system and methods are designed to manage (e.g., control, reduce, eliminate, etc.) harmful organisms by applying electrical energy to the treatment location where they reside to disrupt neurological signals of the target organism or physically alter the target organism's cellular structures.

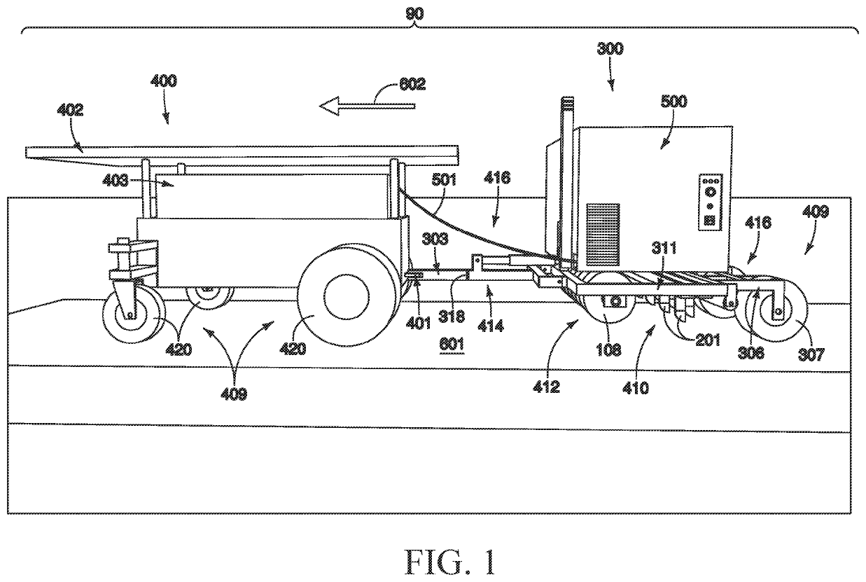

The treatment system may be configured to be stationary or mobile, constructed of steel, aluminum, composite materials (i.e., carbon fiber), plastic or any other structurally-suitable material. One embodiment of a mobile treatment system 90 is shown in FIG. 1 and includes a tow unit and a treatment system that is configured to apply electrical energy to a treatment location as described below. An example mobile treatment system includes a motion assembly including one or more wheels on which to traverse the ground of a treatment location, and may additionally include actuators, hydraulics, mechanics, gravity or other devices by which to orient the electrodes of the apparatus for engagement with the treatment location by changing pitch, roll, and/or yaw, and/or maneuvering or manipulating the electrodes vertically, longitudinally or laterally so as to deliver the electrical energy to the treatment location.

As discussed herein, an example mobile treatment system may be towed by the tow unit or carried by any ground-traversing vehicle or machine that pulls, pushes, carries or otherwise moves and maneuvers the treatment system to traverse a treatment location during delivery of electrical energy to the treatment location.

In one embodiment, the treatment system creates a moving electrical pathway along a swath of the treatment location in order to deliver electrical energy to a relatively large in-situ location, such as an agricultural field, golf green, or sports field. This is achieved according to some embodiments herein by moving electrodes through the material of the treatment location (i.e., soil, root matter, minerals, water, air, etc. or any combination thereof) and relies on the current and voltage carrying capabilities of the treatment location as well as the resistance of the material of the treatment location to complete the circuit of the discharge assembly as discussed further below.

Referring to FIG. 1, a treatment system 90 including a treatment apparatus 300, an associated tow unit 400, and a coupling assembly 414 are shown according to one embodiment. In FIG. 1, the tow unit 400 includes an internal motor (not shown) that propels unit 400 while traversing over ground of a treatment location 601 in a direction of travel 602. The system 90 of FIG. 1 additionally includes a hitch 401 of tow unit 400 and a drawbar 303 of treatment apparatus 300 coupled with the hitch 401.

The treatment system 90 includes a motion assembly 409 that is configured to enable the treatment system 90 to move to traverse over ground of treatment location 601. The illustrated motion assembly 409 includes a wheel carriage 306 and plural tires 307 of treatment apparatus 300 and tires 420 of tow unit 400 to facilitate movement of the apparatus 300 along the treatment location 601. The treatment reduces or eliminates the presence of various organisms or pests present at the treatment location 601. In other embodiments, the treatment apparatus 300 and tow unit 400 are combined into a single unitary apparatus.

In one embodiment, the illustrated unit 400 carries a source of electrical energy in the form of an input power source 403 (e.g., a plurality of rechargeable batteries) and solar panel 402. The batteries may be configured or connected in series or in parallel, or a combination of several sets of batteries may be provided which are connected in series and those sets connected in parallel. Solar panel 402 is used to generate charging electrical energy for charging of input power source 403. Other embodiments of input power source 403 may be used, for example input power source 403 may be in the form of a fossil fuel generator or a power take-off generator. In addition, depleted batteries of the input power source 403 may be replaced with freshly charged batteries as the depleted batteries are recharged. The process of swapping depleted batteries for fresh batteries allows for nearly continuous delivery of electrical energy via treatment apparatus 300 to a treatment location in one implementation.

The treatment apparatus 300 includes a discharge assembly 500, an electrode assembly 410, a preconditioning assembly 412, a positioning assembly 416 and a grooming assembly 308 in the depicted arrangement. Other arrangements of treatment apparatus are possible including more, less and/or alternative components, such as housing an input power source.

Discharge assembly 500 is configured to receive operational electrical energy from input power source 403 by way of interface cables 501. Discharge assembly 500 is configured to control the application of electrical energy to the ground of the treatment location 601 via electrode assembly 410 as discussed below. Electrode assembly 410 includes a plurality of electrodes 201 configured to apply electrical energy to the ground of the treatment location 601 during the traversal of the treatment location 601 by the treatment system 90 in one embodiment.

The illustrated preconditioning assembly 412 is arranged to engage the treatment location 601 prior to the electrode assembly 410 as the treatment apparatus 300 moves along direction of travel 602. Preconditioning assembly 412 includes a plurality of pre-slicing members 108 in one embodiment that are configured to form disruptions in the surface of the ground of the treatment location 601 prior to engagement of the electrodes 201 with the treatment location 601. In one embodiment, the pre-slicing members are configured as rotating discs although the members 108 can have other configurations in other embodiments. In addition, the preconditioning assembly 412 is configured to form the disruptions 604 in the form of grooves in the ground of the treatment location 601. The members 108 are positioned at a plurality of different locations aligned with the electrodes 201 in a lateral direction across a swath of treatment location 601 in a direction substantially perpendicular to the direction of travel 602 of the treatment system 90. Members 108 are metal in one embodiment.

Coupling assembly 414 is configured to couple the tow unit 400 and treatment apparatus 300 together to enable tow unit 400 to tow treatment apparatus 300 during treatment operations. The coupling assembly 414 is also configured to electrically isolate the coupled tow unit 400 and treatment apparatus 300 from one another.

Positioning assembly 416 is configured to adjust the positioning of frame 311 and components mounted thereto relative to the ground of the treatment location 601 in the illustrated embodiment and as discussed further below.

Grooming assembly 308 is configured to condition the surface of the ground of the treatment location 601 following disruption of the ground of the treatment location 601 by the electrode assembly 410 and preconditioning assembly 412.

In example embodiments discussed herein, the discharge assembly 500 is configured to deliver electrical energy to a target volume, such as a volume of soil at a target location in the ground, to reduce or eliminate a variety of pests or other target organisms present in the target volume of soil at the treatment location. In some examples, the discharge assembly is configured to generate and apply pulses of electrical energy having energy profiles that are tailored to impact specific organisms. Example pulses include square waveform pulses of electrical energy that are delivered via electrodes of the treatment apparatus that are engaged with the target volume of soil at the treatment location. In one more specific embodiment, the pulses are generated using a predetermined amount of voltage and current from a capacitive source of high voltage electrical energy and delivered at frequencies known to control harmful organisms at the treatment location.

The discharge assembly 500 may also be referred to as a discharge module and additional details of example treatment systems, treatment apparatuses and discharge assemblies or modules are discussed in the US provisional application recited above as well as co-pending PCT patent application PCT/US2022/037154, filed Jul. 14, 2022, the teachings of which are incorporated herein by reference.

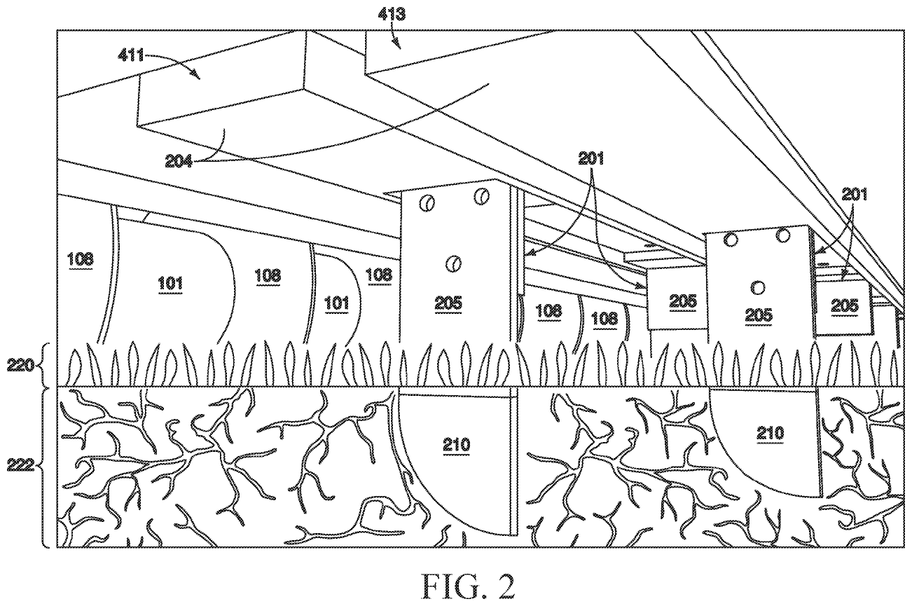

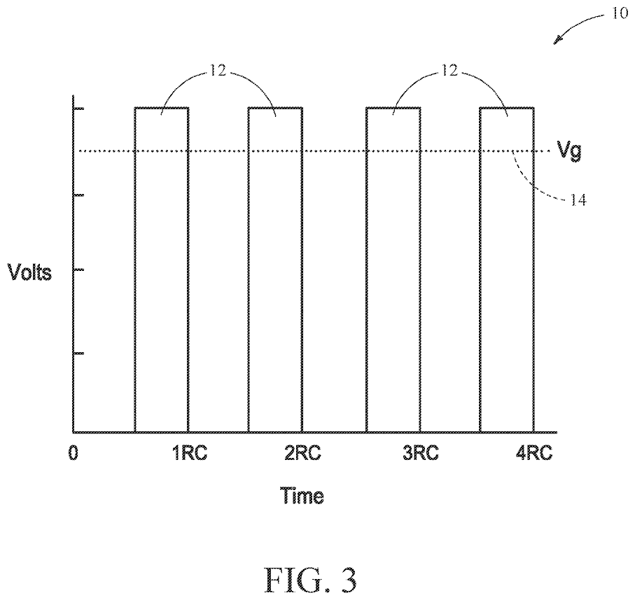

Referring to FIG. 2, exposed, electrically conductive portions 210 of the electrodes 201 are engaged with treatment location 601 below the surface thereof, and electrically isolated portions having a dielectric coating 205 of the electrodes 201 are adjacent to the thatch layer and leaf and crown portions of the plant when electrical energy is supplied to the treatment location 601 in the described embodiment. The electrodes 201 are configured to shield and protect some portions of plants in the treatment location 601 while exposing other portions of the plants to electrical energy.

In the illustrated embodiment, electrode sub-assemblies 411, 413 and upper portions of the illustrated electrodes 201 have a dielectric coating 205 to reduce phytotoxicity during treatment and lower portions of the electrodes 201 are exposed electrically conductive surfaces 210. The dielectric coating 205 is configured to shield at least part of plants in the ground of the treatment location 601 from the electrical energy applied to the ground of the treatment location 601. In particular, the dielectric coating 205 at the upper portion of electrodes 201 shields the thatch layer and leaf and crown portions 220 of the plants while applying the electrical energy to areas in the ground adjacent to the roots of the plants and adjacent soil in a zone 222. As shown, the electrically isolated part of the electrodes 201 are adjacent to and engage the thatch layer and leaf and crown portions 220 of the plants so as to protect the sensitive parts of the plants from the applied electrical energy for treatment by reducing the applied electrical energy to the thatch layer and leaf and crown portions 220 of the plants and delivering the electrical energy to the roots of the plants in zone 222 below.

In one example implementation of treating turf grass, there are no exposed, electrically conductive surface 210 of electrodes 201 visible during treatment as the conductive surfaces 210 of electrodes 201 are beneath the surface of treatment location 601. In addition, the dielectric coating 205 protects the sensitive parts of the turf grass that are in the layer 220 just beneath the surface of treatment location 601. Electrical energy (e.g., generated by discharge assembly 500) is delivered to the roots in zone 222 between electrodes 201 of first sub-assembly 411 and electrodes 201 of second sub-assembly 413 to treat treatment location 601. Accordingly, an increased amount of electrical energy is delivered to portions of the plant including the roots in zone 222 compared with an amount of electrical energy applied to the thatch layer and leaf and crown portions of the plants 200.

The electrodes 201 are configured to conduct a current through the ground at the treatment location 601 in one embodiment. In general, closest adjacent electrodes of opposite polarity of the electrode sub-assemblies 411, 413 conduct currents between one another and through the volume of soil and other matter therebetween during treatment operations. For example, the leftmost electrode 201 of positively-biased sub-assembly 411 may emit a current that is conducted through the ground and other matter at the treatment location to the leftmost electrode 201 of negatively-biased sub-assembly 413.

Utilization of coating 205 according to some embodiments herein is useful for different purposes. First, the exposed, electrically conductive portions of electrodes 201 engage the soil below the thatch layer and leaf and crown portions 220 of the plants concentrating the electrical energy supplied by discharge assembly 300 in the root zone of treatment location 601. By isolating areas of the surface of the electrodes 201, electrical energy from discharge assembly 500 is concentrated below the surface of treatment location 601 where it is desired for the electrical energy to delivered for treatment even when the electrodes 201 are engaged with areas of treatment location 601 where the electrical energy is not to be delivered. For example, the dielectric coating 205 is applied to upper portions of electrodes 201 in the embodiment of FIG. 2 that is configured to isolate and protect the thatch layer and leaf and crown portions 220 of the plants as the electrodes 201 engage the treatment location 601 and deliver electrical energy to areas beneath the protected area of the turf (thatch layer and leaf and crown portions of the plants) of the treatment location 601 where the exposed, electrically conductive, surface area(s) of the electrodes 201 are engaged with the roots and soil of the treatment location 601 where the electrical energy is delivered for treatment. The dielectric properties of the coating 205 prevent the delivered electrical energy from concentrating in the thatch layer and leaf and crown portions 220 of the plants and potentially damaging the sensitive structures of the plants, thus reducing or minimizing the risk of phytotoxicity.

Second, by applying the coating 205 to the electrodes 201 to isolate portions of the electrode surfaces, the uncoated, exposed, electrically conductive surfaces of the electrodes 201 may be optimized to deliver example electrical energy to the treatment location which has a predetermined range of resistance as measured in Ohms. Reducing the area of exposed electrode surface by applying the coating 205 to a larger area of the electrode's overall surface has the effect of raising the amount of resistance in a treatment location. It has been observed that this increased resistance improves the efficient delivery of electrical energy generated by discharge assembly 500 to treatment location 601.

Electrical resistance between the electrodes 201 and the treatment location 601 may be measured and utilized to determine efficient delivery of electrical energy from the discharge assembly of the apparatus 300 to the treatment location 601. The electrical resistance is directly impacted by the amount of exposed, electrically conductive, surface area of the electrodes 201 engaged with the treatment location 601. The combined electrically conductive surface area of all exposed surfaces of one electrode 201 may range between 0.001 square inches and 10,000 square inches to provide an electrical resistance with an example range of 1 to 100k Ohms between the electrodes 201 and treatment location 601. The range of the combined electrically conductive surface area of all exposed surfaces of one electrode 201 may be determined by the resistance, as measured in Ohms, present in a treatment location 601, and varies from treatment location to treatment location based on the characteristics present. For example, the discharge assembly may be configured with different electrode configurations having different electrically conductive surface areas for use in different applications. In a fallow field treatment location, the electrically conductive surface areas of the electrodes are approximately 2.1 square feet or more while the electrically conductive surface areas of the electrodes are approximately 0.11 square feet or more for a sports turf treatment location in some illustrative examples.

Although some example electrodes 201 discussed herein remain in constant engagement with the treatment location 601 by cutting or rolling continuously through the treatment location 601 while being towed by a tow unit 400 and delivering pulses to the treatment location, the treatment apparatus 300 may be configured such that the electrodes 201 can be temporarily, repeatedly inserted (reciprocated) into the treatment location 601 in other embodiments.

A goal of the energy profile generated by some embodiments of the discharge assembly 500 and applied as a moving electrical pathway through a target volume of soil, is managing, controlling or mitigating the function of the target organism through direct contact with an electrical energy profile having selected electrical parameters of voltage, current, pulse width and/or frequency to manage or control pests being treated.

The discharge assembly 500 may generate different types of electrical pulses (e.g., square waveform pulses, bi-polar pulses, bi-phasic pulses) that may have different energy profiles or combinations of different electrical parameters including voltage, current, pulse duration and frequency, resulting in current-induced neurological damage of soil pests, electroporation of supporting cellular structures, or rhabdomyolysis of neurons by electroporation in some examples. Neurological damage can result in impairment of neurological function, causing a failure of one or more behaviors crucial for survival of the soil pest. In one example target organism, plant-parasitic nematodes, neurological damage resulting from the applied electrical energy can impact numerous behaviors including motion necessary for foraging and feeding, or the movement of mouth parts for parasitization, or inhibition of the muscle function necessary for defecation or sexual reproduction. Temporarily impairing or ceasing altogether any one of these behaviors prevents the organism from completing its lifecycle and results in death of the soil pest. Different energy profiles having pulses of different types of pulses and/or different electrical parameters may be used to treat different soil pests as discussed below.

In target organisms which lack a central nervous system, such as the fungal pathogen Fusarium, an energy profile of the pulses of electrical energy is generated by the discharge assembly to cause direct electroporation of the cells of the organism or its propagating forms. This is achieved by applying an appropriate energy profile (e.g., electrical pulse) to the target volume at the treatment location to create a desired electric field, pulsed at a specific frequency, to cause either permanent electroporation or temporary electroporation sufficient for cell lysing to occur, ultimately resulting in the death of the soil pest.

The discharge assembly creates an energy profile using target voltages, pulse shape, pulse duration/width, and/or pulse frequency known to be effective against the target organism. Different types of pulses having different parameters may be used to treat different organisms or pests. As discussed in illustrative embodiments below, these parameters may be selected from reactive energy profiles empirically derived through direct observation, which are based on the size/type of target organism, and factor the volume and characteristics of the target volume of soil of the treatment location. Once a pest to be managed is identified, the discharge assembly 500 is configured to generate an appropriate energy profile including a plurality of different electrical parameters for treatment of the identified pest as discussed further below.

According to some example embodiments of the disclosure described herein, the discharge assembly may implement digital switching (e.g., using FETs, hybrid FETs, MOSFETs or other solid-state switches) to generate pulses of electrical energy from stored energy that have a desired energy profile which, when discharged into the soil or other media, are effective at controlling an identified target soil pest or organism.

Referring to FIG. 3, an example square waveform 10 including a plurality of pulses 12 that may be outputted from the discharge assembly and applied to a treatment location at different time constants is shown.

In some embodiments, a portion of the applied energy content of the square waveform pulse has a voltage above a voltage threshold (Vg) 14 to provide a desired voltage gradient that is known to be effective at controlling pests to the ground at the treatment location to manage the pests. The desired voltage gradient and voltage threshold (Vg) 14 may be different for different pests and is determined by the size/type of target pest as the voltage is applied across two electrodes at a set distance. For example, a grub or insect larvae may only require a 20 V/mm voltage gradient to impact its behavior, while a fungal propagule may require a voltage gradient of 200 V/mm or greater to electroporate its cells. Accordingly, for a given spacing of the electrodes, the voltage threshold Vg is determined that provides the desired voltage gradient across the electrodes for the given spacing of the electrodes. The voltage applied to the ground is a numerator and the electrode spacing is the denominator and the resulting quotient is equal to the resultant voltage gradient. As an example, for a treatment that requires a 40 V/mm voltage gradient with use of an electrode spacing of 10 cm, the voltage threshold (Vg) of the output pulses is 4 KV. Accordingly, in one embodiment, the voltage threshold Vg for a given treatment is determined by the desired or specified voltage gradient to be used for the management of the pest and the spacing of the electrodes.

Average power for square wave pulses can be determined by taking the peak pulse power, multiplying by pulse width (in seconds) and multiplying that product by pulse rate (in pulses per second). Given this, the shorter the pulse width, for the same average power output, the pulse rate is greater which improves the efficacy and efficiency of the treatment by subjecting the target to more changes in electric field intensity with each pulse as there are more pulses per second. The energy of each pulse (“peak power”, or I*E, or watts*pulse duration) and the frequency at which the pulses are applied to the treatment location are the factors that determine efficacy of the tailored energy profiles according to some of the described embodiments.

In one embodiment, the pulses of electrical energy are generated using digital switches as mentioned above. The digital switches are controlled to close to initiate the discharge of electrical energy from the energy storage circuitry (e.g., one or more capacitors) and then open (commutate) to interrupt the pulse, shutting off the flow of energy from the energy storage circuitry leaving the energy storage circuitry in a partially or almost fully charged state enabling quicker recharge of the energy storage circuitry (e.g., approximately 1-10 ns in some embodiments) and resulting in a faster overall pulse cycle compared with some conventional methods, for example that use capacitive decay pulses. A portion of the energy content above the desired voltage threshold 14 may thereby be applied to the treatment location at increased frequencies to achieve a desired level of control of harmful pests or organisms.

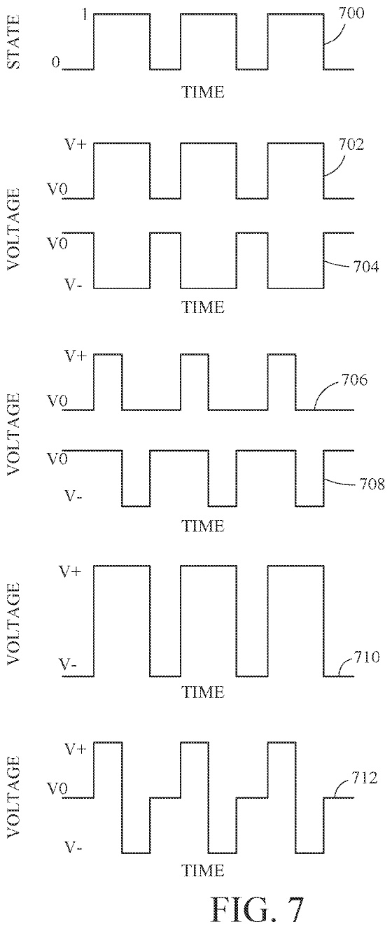

The application of an individual one of the pulses of electrical energy to the ground via the electrodes results in conduction of an electrical current through the ground and between the electrodes. In some of the disclosed embodiments, bi-polar pulses are used to apply electrical energy to the treatment location. The illustrated pulses of FIG. 3 are bi-polar pulses including the summed outputs of positive and negative bi-polar pulses as discussed in further detail below with respect to FIG. 7. Bi-phasic pulses may be used in other embodiments as also discussed below with respect to FIG. 7.

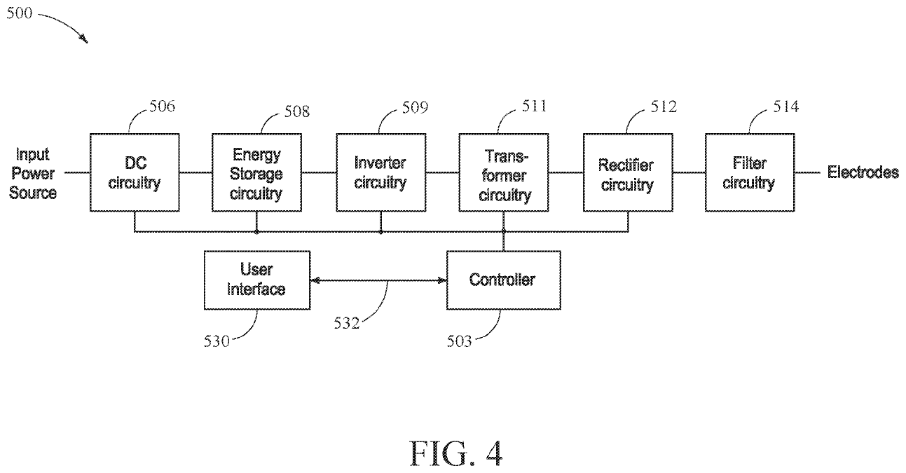

Referring to FIG. 4, electrical components of one embodiment of a discharge assembly (also referred to as a discharge module) 500 are shown. The depicted embodiment of the discharge assembly 500 includes a controller 503, DC circuitry 506, energy storage circuitry 508, inverter circuitry 509, transformer circuitry 511, rectifier circuitry 512, filter circuitry 514 and a user interface 530.

The discharge assembly 500 is configured to receive electrical energy from an input power source which can be different sources of electrical energy in different embodiments (e.g., a land line utility, a standalone generator, a mechanical generator driven by a tractor's power take off (PTO), renewable energy sources and/or batteries).

The electrical energy supplied to the discharge assembly 500 may be direct current (DC) or alternating current (AC) electrical energy. In one AC example, single or multi-phase AC electrical energy is amplified and rectified into high-voltage/high-amperage direct current (VDC), steady state or pulsed DC having a voltage in a range of 10 V to 10 KV, a current in a range of 5 A to 50 KA, and a frequency within a range of 1 Hz to 100 MHz. In one specific embodiment, input power source 403 stores electrical energy and has an output voltage within a range of 48 VDC to 484 VDC and a capacity greater than 3.8 kWh. Other sources of input power delivering electrical energy having different characteristics or parameters may be used in other embodiments.

DC circuitry 506 is configured to receive electrical energy from the input power source and may be implemented as a DC-DC converter in embodiments where the received electrical energy is direct current electrical energy or as a DC power supply in embodiments where the received electrical energy is alternating current electrical energy. Where the input is DC electrical energy, the DC-DC converter receives the energy from the input power source and either bucks (decreases) or boosts (increases) the energy to a voltage that is appropriate for the tailored energy profile outputted from the discharge assembly 500 to a target volume of soil to achieve a desired level of control of harmful organisms. In one embodiment, DC circuitry 506 configured as a DC to DC converter may be implemented as part LB-1071-04-01 40 KW 850V, DC-DC, bi-directional, air-cooled converter available from Zekalabs Ltd. When the input is AC electrical energy, the DC power supply converts the received energy into DC electrical energy at a desired voltage. In one embodiment, DC circuitry 506 receives DC electrical energy in a range from 50 VDC to 800 VDC and outputs DC electrical energy in a range from 100 VDC to 850 VDC or receives AC electrical energy in a range from 120 VAC to 480 VAC and outputs AC electrical energy in a range of 120 VAC to 680 VAC.

The DC electrical energy is provided from DC circuitry 506 to energy storage circuitry 508 that comprises one more devices that operate as intermediate storage circuitry for accumulating and storing the received electrical energy. In one embodiment, the energy storage circuitry 508 includes one or more energy storage capacitor(s) of a capacitor bank. Capacitance of the capacitor bank that stores the electrical energy used to generate the pulses ranges from 1 uF to 1 F. One example capacitor bank may include fourteen 3000 uF capacitors having part number 13396 available from NWL, Inc. and that are arranged in parallel with one another to provide a capacitance of 42,000 uF in one embodiment. The electrical energy may be stored within ranges of 2 V-100 kV and 1 J-50 KJ (e.g., 12 kJ @ 100 pps in one specific example) using the energy storage circuitry 508. The electrical energy is switched and delivered via electrodes of the delivery apparatus to the treatment location in quantities greater than the electrical energy provided by the input power source to the discharge assembly 500. In one embodiment, energy storage circuitry 508 outputs DC electrical energy in a range of 100 VDC to 850 VDC.

The inverter circuitry 509 receives the DC electrical energy from energy storage circuitry 508 and converts the DC electrical energy into AC electrical energy. Inverter circuitry 509 modulates the discharge of the energy from the energy storage circuitry 508 for application to transformer circuitry 511 via a plurality of pulses of high frequency AC electrical energy each comprising a plurality of AC cycles in one embodiment (e.g., each pulse of AC electrical energy has a plurality of cycles at a frequency in a range of 1-500 kHz, and 50-100 kHz in one more specific example).

In one embodiment, controller 503 accesses a value of a pulse width from storage circuitry for a given DC pulse to be applied to the ground. Controller 503 turns inverter circuitry 509 on to output pulses of AC electrical energy each comprising a plurality of cycles of AC electrical energy at the frequency of the inverter circuitry 509 for a duration that corresponds to the accessed value for the pulse width of the DC pulse to be generated and applied to the ground. In one embodiment, the inverter circuitry 509 may modulate pulse width of the cycles of AC electrical energy as a result of monitoring resistance of the ground during the outputting of the pulse of DC electrical energy to control the voltage and/or current of the output DC pulses that are applied to the treatment location as discussed below. In one embodiment, inverter circuitry 509 outputs pulses of AC electrical energy in a range of 100 VAC to 850 VAC.

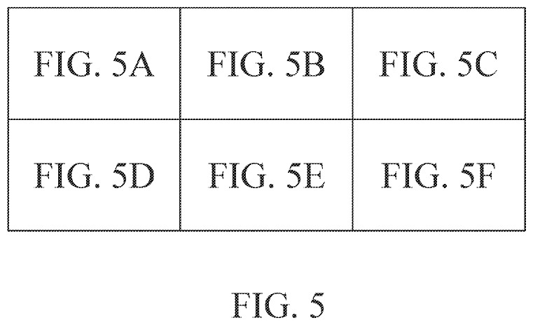

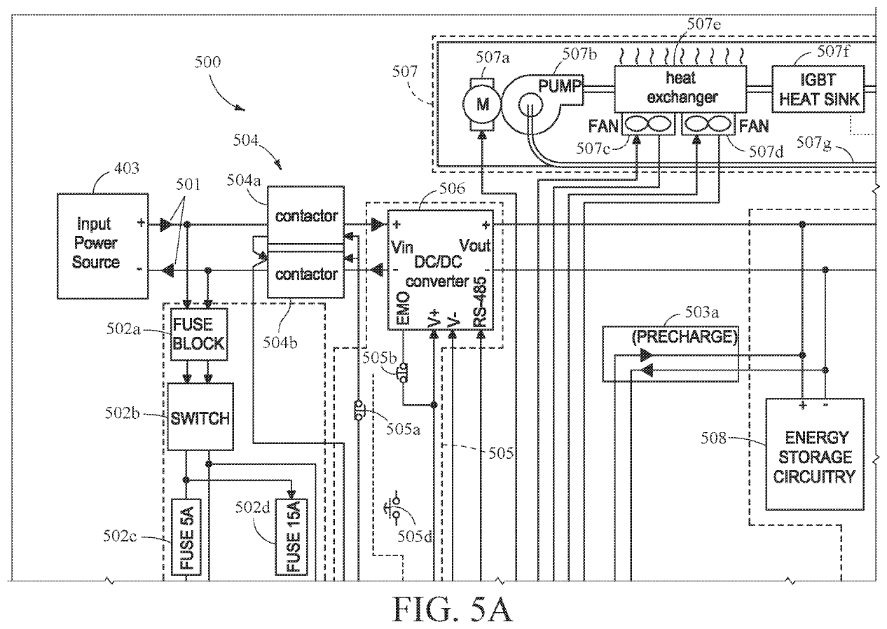

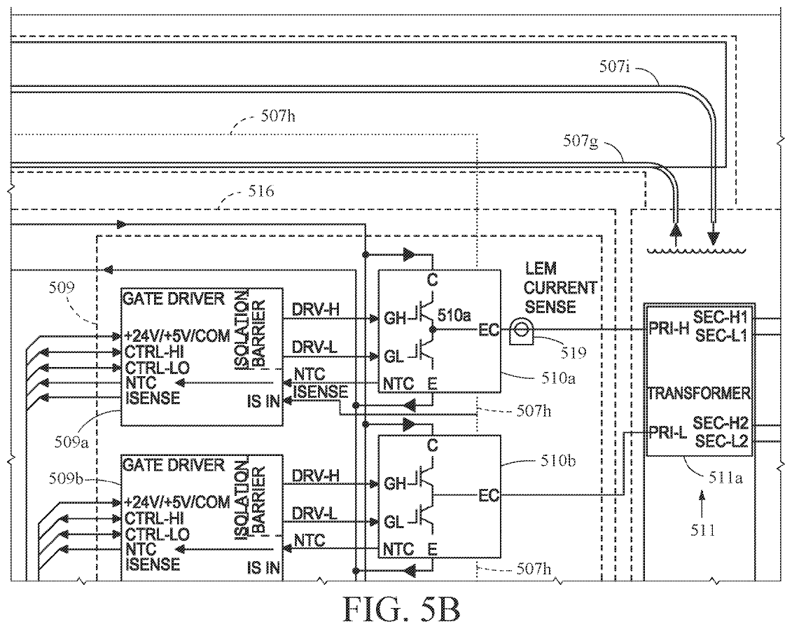

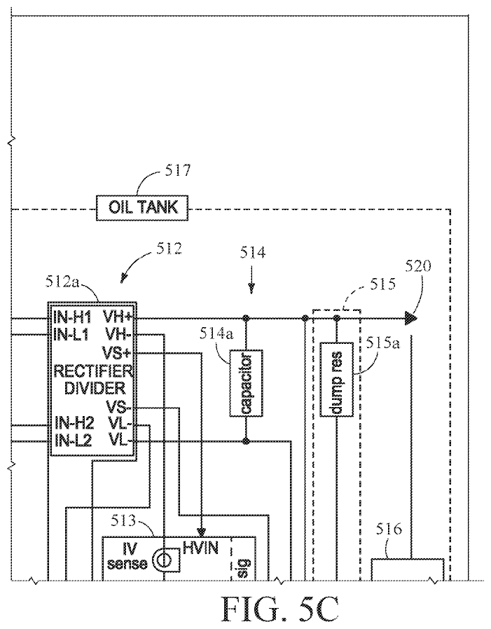

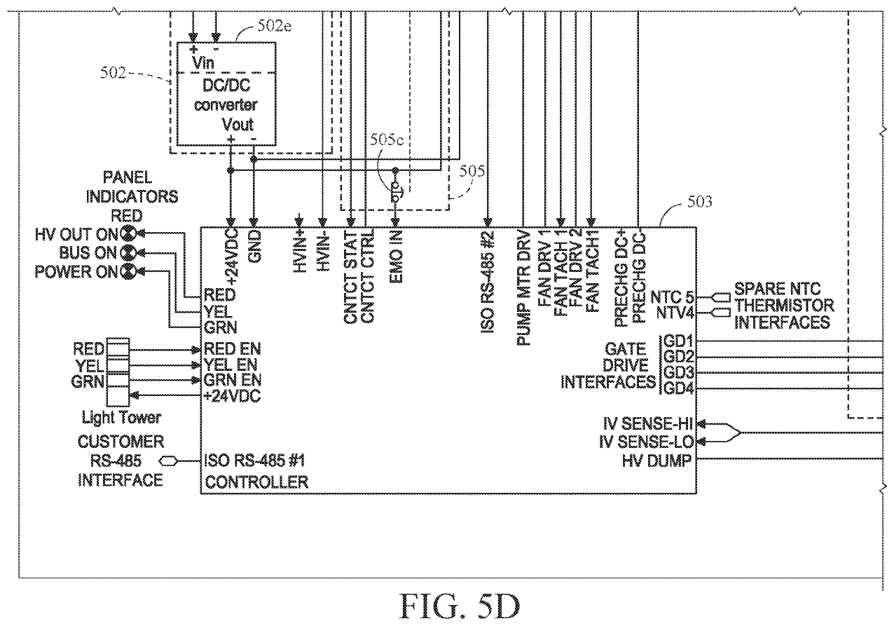

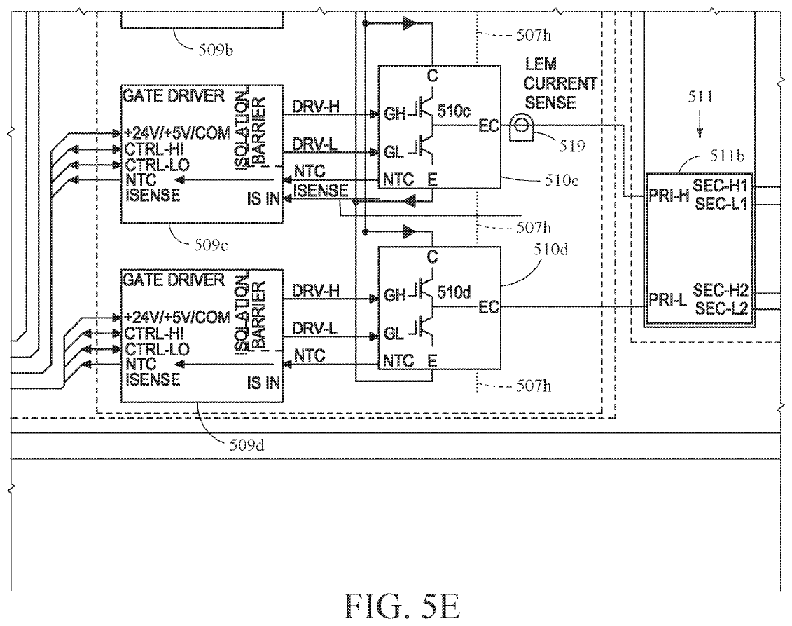

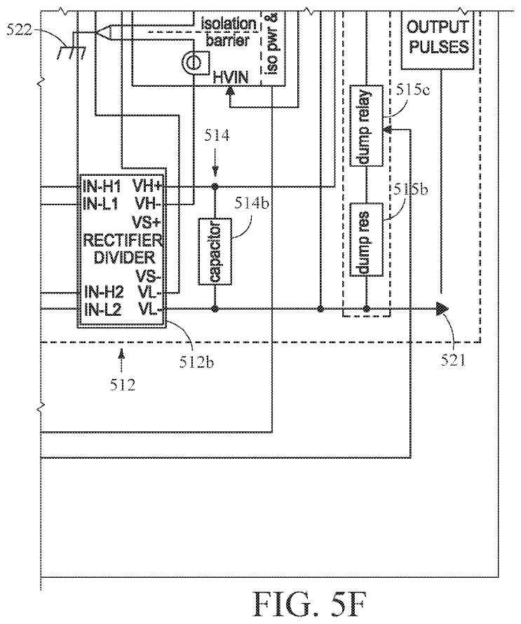

Transformer circuitry 511 receives the pulses of AC electrical energy modulated by the inverter circuitry 509 and converts at least one parameter of the AC pulses (e.g., voltage and current) and outputs the converted AC pulses (also referred to as converted electrical energy) to the rectifier circuitry 512 to generate the DC pulses that are applied to the ground of the treatment location for pest management. Transformer circuitry 511 includes two transformers 511a, 511b in the example embodiment shown in FIGS. 5A-5F and each transformer includes a winding ratio within a range from 10:1-10,000:1 and has a voltage range of 5-1000 kVAC in one implementation. In one more specific embodiment, each transformer has a winding ratio 60:1, 50-100 KHz Nanocrystalline core, Litz wire, stacked, universal-wound secondary, ‘strike pps and kVA’ and voltage of 40 kVAC. In one embodiment, transformer circuitry 511 outputs AC electrical energy in a range of 100 VAC to 34 kVAC.

Rectifier circuitry 512 receives the converted AC pulses of electrical energy from the secondary of transformer circuitry 511 and rectifies the energy into a plurality of DC pulses that are provided to electrodes 201 for application to the ground at the treatment location. In one embodiment, rectifier circuitry 512 outputs DC electrical energy in a range of 100 VDC to 40 kVDC and includes four 10 kV, 1.5 A avg 3 series diodes configured in 4 bridge legs×2 bridges with a time for reverse bias recovery (trr) of 100 ns providing 40 kVDC.

The rectified DC electrical energy outputted from rectifier circuitry 512 is received by filter circuitry 514. In one embodiment, the filter circuitry 514 comprises one or more filter capacitor coupled with the output of the rectifier circuitry 512 to reduce voltage sag of a pulse outputted from discharge assembly 500 and that is discharged into the load as the output voltage of the rectifier circuitry 512 decreases during discharge of the pulse. The filter circuitry 514 is configured to limit the sag of the discharged pulses to less than 20% in one embodiment. The filter capacitors of filter circuitry 514 each have a capacitance of 0.030 uF, voltage of 40 kV, current of 30 A pk and <20% ripple in one embodiment.

In some embodiments, the output of the described discharge assembly 500 at electrodes 201 is a square waveform comprising a plurality of pulses such as shown in FIG. 3. Each square waveform pulse has a 10 microsecond risetime or less (e.g., the amount of time it takes for the pulse of the waveform to go from a specified low voltage value to a specified high voltage value or from 0 V to a setpoint output voltage of the pulse greater than the voltage threshold to provide a desired voltage gradient). The shorter the rise time of a given pulse increases the efficacy and efficiency of the pulse since the amount of applied energy of the pulse that is below the voltage threshold to provide a desired voltage gradient is reduced compared with pulses having increased rise times.

In one embodiment, the pulses of the square wave output each have a pulse duration or width within a range of 100 microseconds to milliseconds measured at full width half maximum (FWHM), a voltage of +/−20 kVDC, and a current within a range of 1-30 A. Square waveforms having other risetimes and pulse durations may be used in other embodiments.

The current conducted through the ground at a treatment location is a function of the voltage and the capacitance of the capacitor(s), filtering, shaping and transforming circuit devices and the resistance of the treatment location. The current conducted through the ground at the treatment location can range from 1 to 10,000 Amps per pulse and voltages of the pulses applied to the ground are in a range of 10 VDC to 100 kVDC in one embodiment. In a more specific embodiment, the voltages of the DC pulses applied to a treatment location are in a range of 100 VDC to 40 kVDC with associated currents in a range of 1-30 Amps.

The values of the pulses may be selected by software of the controller 503 but hardware of the discharge assembly 500 also has physical constraints that are a product of the design and component limitations. Rise time of the pulses is one example of a performance metric that may be hardware limited. For example, during the beginning of a pulse, a plurality of digital switches (e.g., switching circuits 510a, 510b, 510c, 510d discussed below with respect to FIGS. 5A-5F in one embodiment) are driven at their current limit to build energy in the transformer circuitry 511 system as quickly as possible to have a rise time of the pulses as short as the system can perform. The short rise time reduces the amount of energy delivered to the load that is below the desired voltage threshold Vg and increases the efficacy and efficiency of the discharge module 500 compared with arrangements with slower rise times.

As described herein, controller 503 is configured to monitor and control various aspects of the generation and application of the pulses of electrical energy to the ground of a treatment location. In one embodiment, a user interface 530 (e.g., notebook computer) is in communication via a communications link 532 (e.g., WiFi) with the controller 503 enabling an operator to use the user interface 530 to select a desired treatment appropriate for a given application (e.g., fallow field, planted field, sports turf, etc.) and pest to be managed (e.g., nematodes, earthworms, fungal pathogen, etc.).

As discussed with respect to Table A below, pulses of different parameters (e.g., voltage, current, pulse width, frequency) are utilized by the discharge assembly 500 for treatment of different pests to be managed. The user interface 530 receives an input of a user or operator identifying a pest to be managed, and the controller 503 of the discharge assembly selects and sets appropriate values from a plurality of different values for one or more parameters (e.g., voltage, current, pulse width, frequency) of the pulses of electrical energy to be used for a treatment as a result of the receiving the input from the operator that identifies the pest to be managed during a given treatment. In some embodiments, storage circuitry of controller 503 stores a plurality of different values for a plurality of different parameters of the pulses of electrical energy that are applied to the ground for managing different types of pests and controller 503 selects specific values of the parameters for use to manage the pests present with the ground at the treatment location for a given treatment as a result of the input from the user or operator.

Referring to FIGS. 5A-5F, circuitry of one embodiment of a discharge assembly 500 is shown. In the illustrated embodiment, discharge assembly 500 includes a controller power supply 502, a controller 503, DC Circuitry 506 implemented as a DC to DC converter, energy storage circuitry 508, inverter circuitry 509, transformer circuitry 511, rectifier circuitry 512, and filter circuitry 514 among other circuitry.

Controller power supply 502 receives electrical energy from input power source 403 via interface cables 501. Controller power supply 502 provides the electrical energy through a two-pole fuse block 502a to limit the current that is applied to controller 503 (e.g., less than 25 Amps) and feeds that power to two-pole switch 502b that acts as an on/off switch for the discharge assembly 500. When two-pole switch 502b is in the closed or ‘on’ position, it feeds power from the input power source 403 via interface cables 501 through fuses 502c, 502d.

Fuse 502c supplies power to an internal DC to DC converter 502e that converts the electrical energy from the input power source 403 to 24 VDC for use by the controller 503 and DC circuitry 506. In one embodiment, the DC to DC converter 502e is implemented using part number LB-1071-04-01 available from Zekalabs Ltd. Fuse 502d supplies electrical energy to the controller 503 to supply power for the pre-charge circuit 503a to pre-charge the energy storage circuitry 508.

In one embodiment, controller 503 comprises processing circuitry that processes data, controls data access and storage, issues commands, and controls other desired operations of the discharge assembly 500. Controller 503 is implemented using a controller interface PCA and a TI Launchpad TMS320F28379D microcontroller available from Texas Instruments Inc. in one specific embodiment.

In one embodiment, the processing circuitry may process inputs from a user interface to control operations of the treatment system. For example, a user may provide inputs via the user interface to specify an application a pest to be managed for treatment and the processing circuitry of controller 503 may configure the discharge apparatus 500 to treat the identified pest for a given application. Controller 503 sets various parameters of pulses for treatment in one implementation. For example, controller 503 controls operation of contactor circuitry 504, outputs values to DC circuitry 506 to control the voltage of electrical energy outputted from DC circuitry 506 and outputs values to a plurality of gate driver circuits 509a, 509b, 509c, 509d discussed herein to control the generation of AC cycles of electrical energy pulses and parameters thereof (e.g., pulse widths of outputted AC cycles of electrical energy). In addition, controller 503 monitors various operations of discharge assembly 500 including current and voltage of output pulses applied to the ground via IV sense circuit 513.

Processing circuitry is configured to implement desired programming provided by appropriate computer-readable storage media in at least one embodiment. For example, the processing circuitry may be implemented as a microprocessor or other structure configured to execute executable instructions including, for example, software and/or firmware instructions.

Controller 503 may also include data storage circuitry (e.g., memory) that is configured to store programming such as executable code or instructions (e.g., software and/or firmware), electronic data, databases, look up tables (LUTs), or other digital information used by the discharge assembly and may include computer-readable storage media. In one embodiment, the storage circuitry stores a plurality of different values of a plurality of different parameters of the pulses of electrical energy (e.g., voltage, current, pulse width, frequency) for managing different types of pests as discussed below with respect to Table A. Controller 503 is configured to select different values of the parameters to control the generation of pulses of electrical energy that are applied to the ground to manage different types of pests present within the ground at the treatment location for different applications and in response to inputs received from a user or operator via the user interface and which select the pest(s) to be managed in different applications or treatments.

Contactor circuitry 504 includes two primary contactors 504a, 504b that are controlled by the controller 503 and that selectively connect the electrical energy supplied by the input power source 403 to the DC circuitry 506 and energy storage circuitry 508 to charge the energy storage circuitry 508. The contactors 504a, 504b are activated after the pre-charge circuit 503a has charged the voltage in the energy storage circuitry 508 to a voltage equal to the input power source 403 in one embodiment.

The Emergency Mains Off (EMO) circuit 505 includes a control circuit 505d with plural switches that are in a series loop and control the contactors EMO 505a, 505b and 505c. Contactor 505a provides power to the primary contactors 504a and 504b so that in the event of an emergency the system can disconnect from the input power source 403. Contactor 505b provides power to EMO circuit 505 from DC circuitry 506. Contactor 505c provides power to an input to controller 503 that informs the control system of the state of EMO circuit 505. An operator may enable the EMO circuit 505 to disconnect the input power source 403 from the discharge assembly 500 in one implementation.

DC circuitry 506 is supplied power from the input power source 403 through primary contactors 504a, 504b. The DC circuitry 506 converts the power from the input power source 403 to a voltage value supplied by the controller 503 and charges the energy storage circuitry 508. Controller 503 may provide voltage values of electrical energy to be outputted from DC circuitry 506 depending on the particular type of treatment location being treated by the treatment system (e.g., fallow field, sports turf, etc.). In illustrative examples, controller 503 controls DC circuitry 506 to output DC pulses in a range of 2 to 40 kVDC for treatment of sports turf, 5 to 20 kVDC for treatment of vegetable row crops, or 10 to 50 kVDC for treatment of fallow soil.

As mentioned above, the energy storage circuitry 508 is pre-charged by the controller 503 in one embodiment. Once the energy storage circuitry 508 has the same voltage as the input power source 403, the DC circuitry 506 provides an output voltage determined by a value provided by controller 503 to charge the energy storage circuitry 508 for storage of electrical energy for use in generating the pulses applied to the treatment location.

In one embodiment, the DC circuitry 506 receives electrical energy at a voltage of 48 VDC and outputs electrical energy at a voltage of 800 VDC.

The DC circuitry 506 is also a component in the EMO circuit 505 where, in the event of an activation of the EMO circuit 505, the DC circuitry 506 stops supplying power to the energy storage circuitry 508 and isolates the energy storage circuitry 508 from the input power source 403 in addition to the EMO circuit 505 deactivating the primary contactors 504a, 504b.

In one embodiment, the energy storage circuitry 508, being fully charged by the DC circuitry 506, applies electrical energy to inverter circuitry 509 by solid copper buss bars. In one embodiment, inverter circuitry 509 includes a plurality of gate driver circuits 509a, 509b, 509c, 509d and a plurality of switching circuits 510a, 510b, 510c, 510d that may be implemented as dual half bridge circuits including a plurality of insulated-gate bi-polar transistors (IGBTs). In one specific embodiment, the switching circuitry 510 is implemented with four bridge legs using four 1700 V, 225 A, 8.0 mΩ, silicon carbide, half-bridge modules having part number CAS300M17BM2 and available from Wolfspeed, Inc.

In example embodiments described herein, controller 503 controls the inverter circuitry 509 to selectively discharge electrical energy stored in the energy storage circuitry 508 to generate a plurality of pulses of electrical energy that are applied to the ground. In the embodiment of FIGS. 5A-5F, controller 503 controls the application of AC gate drive or control signals from gate driver circuits 509a, 509b, 509c, 509d to gates of switching circuits 510a, 510b, 510c, 510d to control opening and closing of switching circuits 510a, 510b, 510c, 510d to selectively conduct electrical energy from energy storage circuitry 508 and generate and output a plurality of cycles of the AC electrical energy to transformer circuitry 511 for generation of the square waveform or other pulses that are applied to the ground at the treatment location. In one embodiment, the gate driver circuits 509a, 509b, 509c, 509d generate the AC drive signals including a positive voltage (e.g., +25 V) to close switching circuits 510a, 510b, 510c, 510d and a negative voltage (e.g., −17 V) to open switching circuits 510a, 510b, 510c, 510d.

The controller 503 turns on the inverter circuitry 509 for a length of time determined by a desired pulse width of a DC pulse to be output from discharge assembly 500 and applied to ground and controls the gate driver circuits 509a, 509b, 509c, 509d to output the diving signals for the determined length of time corresponding to the pulse width of the DC pulse. For example, controller 503 accesses a value of a pulse width of a DC pulse to be applied to the ground (e.g., see Table A) and then controls the inverter 509 to output a pulse the AC electrical energy for the same length of time as the pulse width of the DC pulse to be generated.

In some embodiments, the energy storage circuitry 508 stores electrical energy at a voltage greater than the voltage threshold (Vg) of electrical energy to provide a desired voltage gradient as discussed above with respect to FIG. 3 to be applied to the treatment location for management of the pests. Controller 503 accesses a value of a pulse width of a DC pulse to be outputted from discharge assembly 300 to manage a given pest (e.g., from Table A below) and controls the inverter circuitry 509 to output AC electrical energy for the length of time corresponding of the value of the pulse width for the DC pulse to be formed and applied to the ground.

The values of the pulse widths of the DC pulses that are to be applied to the ground are selected to maintain a voltage of the energy storage circuitry 503 at a level that is at least substantially equal to or greater than the voltage threshold being utilized to provide a desired voltage gradient to manage the pests for a given treatment and during the generation and application of the pulses to the ground in one implementation. This enables the energy storage circuitry 508 to be quickly recharged to an increased voltage (e.g., maximum voltage Vmax) over the voltage threshold Vg which enables the DC pulses to be output and applied to the ground of the treatment location at increased frequencies thus improving the efficacy and efficiency of the management of the pests.

In one embodiment, the pulses of electrical energy that are applied to the ground each have a voltage greater than the voltage threshold Vg for substantially an entirety of the pulse width of the individual pulse applied to the ground (e.g., except for the rise time at the initiation of a given pulse) as shown FIG. 3. The switching circuits 510a, 510b, 510c, 510d remain in an off state following the generation and application of a DC pulse having a desired pulse width to the ground and prior to the voltage of the energy storage circuitry 508 and the DC pulses dropping below the voltage threshold Vg in one embodiment.

When a treatment pulse is initiated by the controller 503, the gate driver circuits 509a, 509b, 509c, 509d produce AC drive signals (e.g., within a frequency range of fixed 50-100 kHz) that allow the switching circuits 510a, 510b, 510c, 510d to selectively and repeatedly enter a conductive state to output a plurality of cycles of AC energy (e.g., within a frequency range of 50-100 kHz) from energy storage circuitry 508 to transformer circuitry 511 for a period of time corresponding to the pulse widths of the DC pulses that are applied to the ground.

Current sensors 519 monitor the current of cycles of AC electrical energy outputted from the switching circuits 510a, 510b, 510c, 510d and may be used by gate driver circuits 509a, 509b, 509c, 509d to avoid conduction of excessive current using switching circuits 510a, 510b, 510c, 510d, for example during the generation of a few initial cycles of AC electrical energy from inverter circuitry 509 for a given DC pulse that is applied to the ground at the treatment location.

In the above-described embodiment, the switching circuits 510a, 510b, 510c, 510d are selectively controlled to close and open to generate cycles of AC energy that are applied to transformer circuitry 511 for the generation of a DC pulse to be applied to the ground. Following the generation of a DC pulse having a desired pulse width for a given pest to be managed (e.g., see Table A), the controller 503 and is programmed to control the gate driver circuits 509a, 509b, 509c, 509d to open the switching circuits 510a, 510b, 510c, 510d and which corresponds to discharge of the applied energy content from the initial maximum voltage level Vmax for the DC electrical pulse (e.g., a maximum capacity of the energy storage circuitry 508) to the desired voltage threshold (Vg) below which energy discharged from the energy storage circuitry is less effective. Upon opening of switching circuits 510a, 510b, 510c, 510d, the energy storage circuitry 508 recharges to Vmax or a set level above the voltage threshold (Vg) in preparation for the discharge of a subsequent DC pulse. Accordingly, in some embodiments, the energy storage circuitry 508 remains at lease substantially at, above or slightly below the voltage threshold Vg during the generation and application of DC pulses to the treatment location via the electrodes of the treatment apparatus.

The forced commutating of switching circuits 510a, 510b, 510c, 510d enables generation and application of pulses to the ground having shorter pulse widths compared with the use of capacitive decay pulses which allows for higher frequency pulsing for the same amount of average power of the treatment.

As mentioned above, some embodiments of the discharge assembly control a given DC pulse applied to the ground by allowing only the applied energy of the pulses above the voltage threshold Vg of the energy stored in the energy storage circuitry to be discharged in each DC pulse leaving a majority of the stored energy in the energy storage circuitry which shortens the recharge time of the energy storage circuitry. The above-recited control of the DC pulses allows for higher pulse frequencies and ensures the energy of each pulse is at least substantially at or above the desired voltage threshold Vg being used to achieve the desired level of control of the target organism according to one embodiment.

The forced commutation process described above allows generation of DC pulses of electrical energy for application to the ground that are analogous to a digital signal with a tailored energy profile and a distinct amplitude. By applying the most effective electrical energy of each DC pulse discharge (i.e., the applied energy) at a wider range of frequencies, a more finely-tuned, high-fidelity waveform of DC pulses can be achieved according to some embodiments of the disclosure.

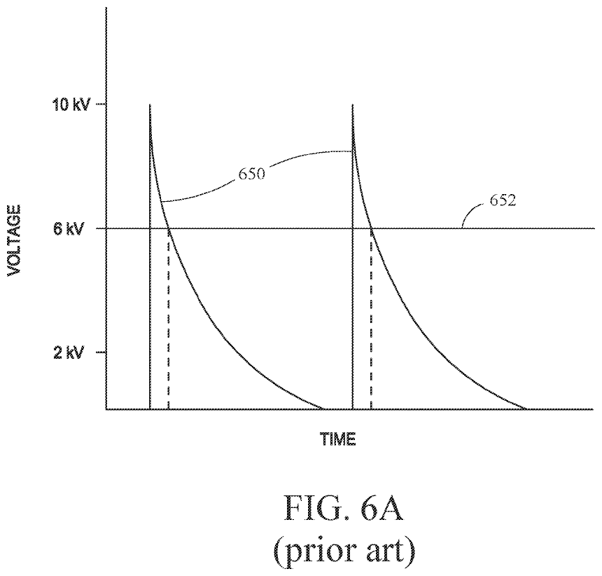

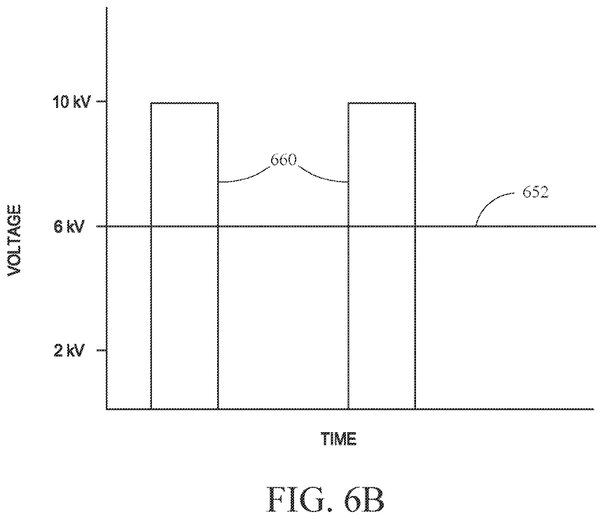

Additionally, the use of force commutated switching circuits 510a, 510b, 510c, 510d enable development of a square wave DC pulses that increases efficiency by ensuring a portion of the applied energy content of the DC pulse is above the efficacy voltage threshold Vg corresponding to the desired voltage gradient to be applied to the treatment location. In comparison to capacitive decay waveforms, for example, a square wave output increases efficacy and efficiency for the same amount of total energy delivered to the ground as discussed below with respect to FIGS. 6A and 6B.

When turned on by controller 503, the inverter circuitry 509 outputs a plurality of cycles of AC electrical energy for application to transformer circuitry 511. Transformer circuitry 511 includes two transformers 511a, 511b that are each implemented as a dual transformer having a common core that is housed in an oil tank 517 in the described embodiment. The cycles of AC energy from inverter circuitry 509 are applied to the primary windings of transformers 511a, 511b of transformer circuitry 511 which provides voltage an current conversion of the electrical energy. Transformers 511a, 511b each include magnetically coupled primary and secondary windings which convert voltage and current of the AC cycles from the inverter circuitry 509 and apply the converted AC cycles to rectifier circuitry 512.

In one embodiment, rectifier circuitry 512 includes dual bridge rectifier dividers 512a, 512b which may be implemented using part number K100UF available from Voltage Multiplier Inc. The dual bridge rectifiers 512a, 512b rectify the AC cycles into a DC output pulse. The rectifier dividers 512a, 512b are referenced to a common node 522 in the illustrated embodiment.

In the illustrated example, the output of the secondaries of transformers 511a and 511b are rectified by the rectifier circuitry 512 and the resulting center tap at the IV sense circuit 513 creating bi-polar DC pulses or bi-phasic DC pulses that are outputted and applied to the ground in example embodiments. The DC pulses from the dual bridge rectifiers 512a, 512b are also applied to the IV sense circuit 513 that monitors voltage and current of the DC pulses and generates and communicates voltage and current values to the controller 503 that are indicative of the DC pulses outputted from the rectifier circuitry 512. Controller 503 monitors current and voltage from IV sense circuitry 513 to monitor resistance of the ground during the application of DC pulses of electrical energy to the ground and may be used to adjust operations of the inverter circuitry 509 as discussed below in one embodiment. IV circuit 513 is also coupled with common node 522.

The DC pulses outputted from the dual bridge rectifiers 512a, 512b are further applied to filter circuitry 514 including plural filter capacitors 514a, 514b in the illustrated embodiment. The filter capacitors 514a, 514b are connected in parallel with the output of the discharge assembly 500 and provide filtration, increased fidelity and reduced ripple of the outputted DC pulses sent to the electrode assemblies of the delivery apparatus in one embodiment.

The dual bridge rectifiers 512a, 512b and filter capacitors 514a, 514b are connected in parallel with high voltage dump circuitry 515. Dump circuitry 515 provides a dissipation path for the DC output of the dual bridge rectifiers 512a, 512b and includes dump resistors 515a, 515b and a dump relay 515c controlled by the controller 503. In one example, dump circuitry 515 provides safe discharge of electrical energy from the energy storage circuitry 508 in the event of a shut-down or other appropriate reason for discharging energy stored in the energy storage circuitry 508, such as a communication failure with the user interface, a control error in the gate driver circuits, an error in the inverter circuitry, an error in the voltage supplied by the input power source, a controller watchdog timer time out (internal communication error), or user initiated emergency stop in some examples.

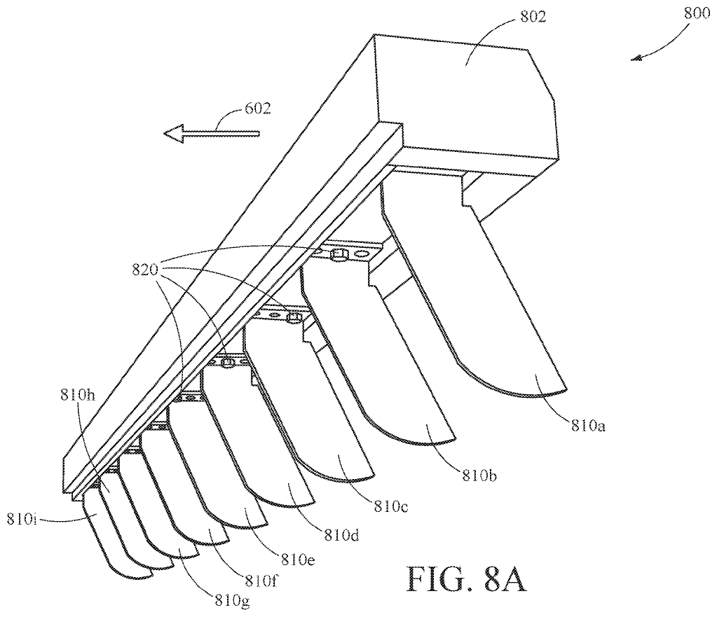

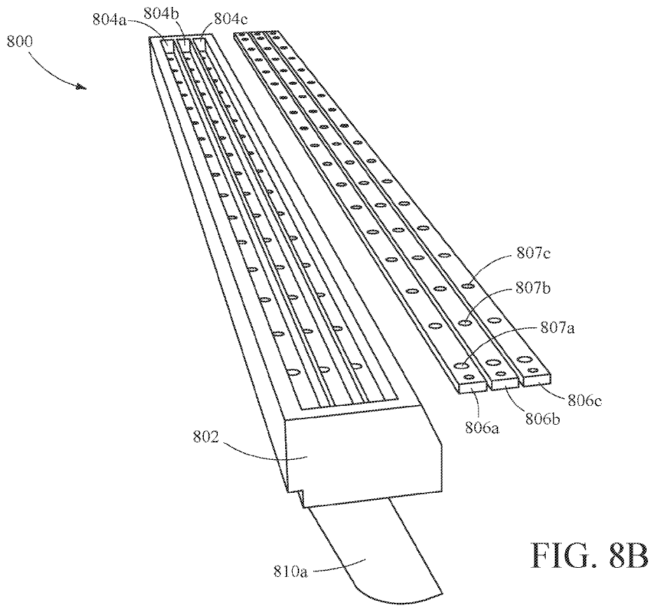

In one embodiment, dual bridge rectifier 512a outputs a plurality of positive voltage pulses with respect to a common voltage reference and dual bridge rectifier 512b outputs a plurality of negative voltage pulses with respect to a common voltage reference to form a plurality of DC pulses that are applied to the ground (see FIG. 7 and discussion below). The positive and negative pulses are applied to respective positive and negative outputs 520, 521 for application to the ground at the treatment location in one embodiment. Positive and negative outputs 520, 521 may be coupled with electrodes 201 shown in FIG. 2 or electrodes 810 shown in FIGS. 8A and 8B in example embodiments.

When observed from the load, the positive and negative voltage pulses are combined to form synchronized bi-polar pulses or asynchronized bi-phasic pulses in example embodiments and as discussed below with respect to FIG. 7. For example, in some embodiments, the positive and negative pulses are synchronized with respect to time to generate pulses of direct current electrical energy comprising square waveform bi-polar pulses, while in other embodiments, the positive and negative pulses are not synchronized with respect to time to generate pulses of direct current electrical energy comprising square waveform bi-polar and bi-phasic pulses.

As mentioned above, both voltage (e.g., voltage divider on the rectifier divider circuits 512a, 512b) and current of the DC pulses may be measured by IV sense circuitry 513 and provided to a multiplexer of the controller 503 for monitoring of the DC pulses applied to the ground in one embodiment.

The described example embodiments allow exceptionally high levels of current to flow within the ground during the application of the DC pulses (e.g., up to 3600 Amps to flow between respective positive and negative outputs 520, 521 when closed in the embodiment of FIGS. 5A-5F) and switching circuits 510a, 510b, 510c, 510d have on/off rates in the sub-microsecond range.

Oil cooling system 507 is a closed loop system in the illustrated embodiment and includes a motor 507a that is controlled by the controller 503. The controller 503 controls the motor 507a to provide cooling of switching circuits 510a, 510b, 510c, 510d (e.g., IGBTs in the illustrated embodiment) during the application of electrical energy to a target volume of soil. The motor 507a is mechanically connected to an oil pump 507b that circulates oil from a transformer tank 517, via rigid copper piping 507g, through a heat exchanger 507e which is mounted in thermal contact 507h with a heat sink 507f to cool switching devices 510a, 510b, 510c, 510d. Rigid copper piping 507i returns the oil to the oil tank 517. Fans 507c, 507d are controlled by the controller 503 and force air through the heat exchanger 507e when a maximum thermal value supplied by the controller 503 is detected and the fans 507c, 507d are controlled to stop operation when a minimum thermal value supplied by the controller 503 is detected in one embodiment.

The energy storage circuitry 508 and inverter circuitry 509 are housed in a protective enclosure 516 in some embodiments where increased amounts of electrical energy are stored in the energy storage circuitry 508.

Electrical resistance (e.g., in Ohms) of the ground and soil at a treatment location may be measured and monitored by the controller 503 of discharge assembly and utilized to determine efficient delivery of electrical energy from the discharge assembly of the apparatus to the treatment location. The electrical resistance is directly impacted by the amount of exposed, electrically conductive, surface area of the electrodes that is engaged with the treatment location as discussed above.

A unique characteristic of applying electrical energy to a large, in-situ location, such as an agricultural field, golf green, or sports field, is the varying resistance found within the volume of material at the treatment location as the electrodes are moved through the treatment location and new electrical pathways are created. In some embodiments, the current and voltage carrying capabilities of the treatment location is utilized to facilitate a discharge of current through ground at the treatment location between one electrode provided at one voltage polarity (or bias) to one or more electrodes that adjacent to the one electrode and are provided at a different polarity (or bias), while relying on the electrical resistance of the treatment location to prevent the discharges generated by the discharge assembly from causing an arc event while the electrodes are engaged with the treatment location. This example configuration allows a tailored energy profile having a specified voltage, current, pulse width and frequency to be generated by the discharge assembly and pulsed across the target volume at the treatment location which may include one or more of soil, water, growing media, air, fallow or where plants, weeds, roots, etc. may be present in between the electrodes.

A treatment location such as turf or agricultural field will have different mineral composition, moisture content, root matter density, etc. that varies from square meter to square meter as the delivery apparatus moves the electrical pathway created by its electrodes through the target volume of soil at the treatment location. For example, a golf green is a homogenous, contrived environment constructed in a very specific way to support the health of the turf, yet the electrical resistance of the combined soil, root matter, minerals and other components that make up the material beneath the playing surface varies widely, in some cases several thousand Ohms in only a few feet. This inconsistency impacts the current and voltage carrying capabilities of the ground at different treatment locations. These changing characteristics all affect resistance which is an important factor in applying the necessary energy profile to target and manage soil-borne organisms.

The typical treatment medium for the discharge assembly is soil, with or without a plant or cropping system present. This environment presents multiple current pathways for the electrical energy applied to the treatment location. Each of these paths has a given impedance (resistance) that can also be represented by an aggregate measurement of soil electrical conductivity. Electrical conductivity measurement is an electrolytic process that takes place principally through water-filled pores. Cations (Ca2+, Mg2+, K+, Na+, and NH4+) and anions (SO4 2−, Cl−, NO3−, and HCO3−) from salts dissolved in soil water carry electrical charges and conduct an electrical current. Consequently, the concentration of ions is the primary factor that determines the electrical conductivity of soils. The presence of plant root structures and other organic materials can also provide an alternate current path independent of the electrolytic pathways in soil pores.

A determining factor in the efficacy and efficiency of the energy profile is to control and adjust the output of the discharge assembly for the various pathways that present themselves as the pulses of electricity are being delivered to a given treatment location.

Given that resistance is equal to the voltage divided by the current, a voltage is developed across the output of the discharge assembly as a given amount of current is applied from the treatment apparatus to the ground having a given resistance (load resistance (R)). Controller 503 monitors parameters (e.g., voltage and current) of the DC pulses of electrical energy that are applied to the ground via IV sense circuit 513 to determine resistance of the ground at the treatment location.

In one embodiment, controller 503 adjusts operations of the inverter circuitry 506 including control of the generation of the cycles of AC electrical energy as a result of the monitoring of DC pulses as discussed below. The monitoring and adjustment are used to provide and maintain conduction of peak current of the DC pulses of electrical energy that are applied to the treatment location in one embodiment.

In one embodiment, the discharge assembly detects the resistance of the load, pulse-to-pulse, and controls the output of the electrical energy applied by the electrodes to compensate for varying resistance and maintain desired voltage and current to deliver a uniformly effective energy profile throughout the treatment location.

In one embodiment, the controller 503 calculates resistance from sensed voltage and current and adjusts the characteristics of the generated DC pulses that are applied to the ground every 50 micro-seconds in response to the changing soil conductivity to generate the selected energy profile chosen based on the target organism and characteristics of the treatment location. This ensures the energy profile is consistently above a minimum voltage threshold Vg of efficacy to provide a desired voltage gradient to the treatment location irrespective of changes in soil conductivity (e.g., continuously apply electrical energy pules above a voltage threshold that provides at least the desired voltage gradient to the treatment location).

The controller 503 changes or adjusts the pulse widths of the driving signals outputted from gate driver circuits 509a, 509b, 509c, 509d to change or control the pulse widths of the AC cycles of the pulses of AC electrical energy that are outputted from inverter circuitry 509 and applied to transformer circuitry 511 as a resulting of the monitoring in one embodiment. The pulse widths of the cycles of the pulses of AC electrical energy outputted from the inverter circuitry 509 may be varied as a result of monitoring current and voltage of the output DC pulses applied to ground and that are indicative of resistance of the ground. Variation of the on-time of the switching circuits 510a, 510b, 510c, 510d according to gate drive signals adjusts the current in the primaries of the transformer circuitry 511. The changing of the pulse widths of the AC cycles of electrical energy changes or adjusts one or more parameter (e.g., voltage or current) of the DC pulses of electrical energy that are applied to the ground as a resulting of the monitoring in one embodiment.

Controller 503 may use different control methods including current control or voltage control in different embodiments that are described below. Important to the efficacy of the treatment is the maintenance of a consistent output throughout the target volume of soil being treated to apply or deliver an effective energy profile to the entire target volume of soil at the treatment location to control the target organism(s) therein.

In some embodiments, voltage control is utilized where the voltage output at the electrodes is measured and the current is changed as the load at the treatment location changes to maintain the voltage regulation setting. As the resistivity of the soil changes while the delivery device moves through the soil at the treatment location, the output voltage change is measured by the system and the current output is adjusted to maintain the desired set output value. The voltage of the output DC pulses is measured via IV sense circuit 513 and compared to a maximum voltage setting of the energy profile for a given pest to be managed (e.g., see Table A). If the measured voltage is greater than the set maximum voltage, controller 503 reduces the pulse widths of the gate drive or control signals provided to the gate driver circuits 509a, 509b, 509c, 509d shortening the pulse widths of the AC cycles outputted from the inverter circuitry 509 to reduce the current of the DC pulses. The pulse widths of the drive values are reduced until the measured voltage of the output DC pulses is substantially at the threshold set by the energy profile to maintain a substantially constant voltage of the DC pulses applied to the ground in one embodiment. In addition, the pulse widths of the gate drive signals may be increased to increase the voltage of the DC pulses as needed.

In another embodiment, current control is utilized where the current of the DC pulses outputted from the discharge assembly 500 are directly measured via IV sense circuitry 513 and compared to a maximum current setting of the energy profile (e.g., see Table A). If the measured current is greater than the maximum current setting, controller 503 reduces the pulse widths of the drive values provided to the gate driver circuits 509a, 509b, 509c, 509d shortening the pulse widths of the AC cycles outputted from the inverter circuitry 509 to reduce the voltage of the DC pulses. The pulse widths of the drive values are reduced until the measured current of the output DC pulses is substantially at the threshold set by the energy profile to maintain a substantially constant current of the DC pulses applied to the ground in this embodiment. In this instance, as the load changes and the current stays constant, voltage will fluctuate as the soil resistance changes. In addition, the pulse widths of the gate drive signals may be increased to increase the current of the DC pulses as needed.

The above-described measurements and adjustments made by controller 503 compensate for continuous variations in the load as the treatment is delivered. In one embodiment, the analog outputs from the voltage and current sensors of IV sense circuit 513 are connected to analog to digital converters and multiplexed for reading by the controller 503. Look up and conversion tables in data storage circuitry of controller 503 are referenced and the drive values applied to gate driver circuits 509a, 509b, 509c, 509d are modulated to control the energy profile to the desired output in example embodiments.

Some embodiments of the disclosure deliver an at least substantially consistent, uniformly effective profile of electrical energy known to be effective against target organisms despite variances in the treatment locations. In areas of the treatment location that have high resistance, voltage tends to rise and current tends to drop, whereas in areas of low resistance, voltage tends to drop and current tends to rise. During treatments in soil of increased resistance, usage of an energy profile with a higher voltage resulted in a more effective outcome of controlling the target organisms. In some examples, areas of bare dirt where turf grass could not grow because of high nematode populations, those areas would be several times more resistive than areas where turf was denser, causing the discharge assembly to output more voltage at a lower current. The applied energy profile is effective at reducing nematode populations allowing turf grass to recover. In the above-described example embodiments, the e discharge assembly compensates for ever-changing resistance in the soil of the target volume at the treatment location to provide effective treatment.

In one embodiment, the initial drive parameters are set by a peak current value of the controller 503 based on the selected energy profile to be used to treat a given soil pest at a given treatment location. The peak current value is translated by the controller 503 into a value that is passed to the gate driver circuits 509a, 509b, 509c, 509d. The gate driver circuits 509a, 509b, 509c, 509d use the received values to select a regulated on-time of dual half bridge switching circuits 510a, 510b, 510c, 510d to achieve the peak current setting requested by the energy profile according to one embodiment. This value also acts as an upper limit of the current of electrical energy that is conducted between the electrodes. As discussed herein, controller 503 reduces the drive values applied to gate driver circuits 509a, 509b, 509c, 509d to comply with the peak current and other limits (e.g., voltage, current, temperature) that may be set by the chosen energy profile.

In other embodiments, controller 503 may interrupt the output temporarily (e.g., when an arc is detected) and then resume output as the treatment apparatus moves into a different area of the target volume being treated. The measured values in voltage are greater than the measured values in current in typical pulses. When an arc is formed, the apparent load presented to the discharge assembly is a short causing the voltage and current to invert. When this change is detected, the treatment system stops all application of electrical energy to the treatment location and an arc fault is registered in one embodiment.

In one embodiment, the gate drive values control the time the switching circuits 510a, 510b, 510c, 510d are on and are set by characterization of the circuit dynamics and characteristics of transformer circuitry 511. In one embodiment, the characterization is used to establish a look up table (LUT) of digital-to-analog conversion values that controller 503 references when sending drive requests or control signals to the gate driver circuits 509a, 509b, 509c, 509d. These drive requests (gate on time) are limited by the measured current output to ensure the output specifications of the switching circuits 510a, 510b, 510c, 510d are not exceeded until sufficient energy has been received in the transformers 511a, 511b and the controller 503 generates the drive values of the gate on times of the switching circuits 510a, 510b, 510c, 510d. The gate operating voltages of switching circuits 510a, 510b, 510c, 510d can be a range of voltages that are set by the charge state of the energy storage circuitry and can range from 10 VDC to 10 kVDC. A more specific example range of gate operating voltages is 500 VDC to 750 VDC.

The leading-edge profile of the pulses of electrical energy is determined by the reactive components of the primary side of the transformers 511a, 511b. In the first few pulses (e.g., 1-10) outputted from inverter circuitry 509 for the generation of given pulse to be applied to the target volume of soil at the target location, the switching circuits 510a, 510b, 510c, 510d reach their current limits as monitored by current sensors 519 and are switched off by gate driver circuits 509a, 509b, 509c, 509d. An initial duty cycle of the gate drive signals applied to switching circuits 510a, 510b, 510c, 510d reach may be 40-60%.

This continues until the time enough energy is in the primaries of the transformers 511a, 511b and the controller 503 thereafter modulates the on time of the gates of the switching circuits 510a, 510b, 510c, 510d via the gate driver circuits 509a, 509b, 509c, 509d to provide DC pulses of desired voltage and current. Once the system builds energy in the transformers 511a, 511b and switching circuits 510a, 510b, 510c, 510d are no longer operating at their current limit, the gate drive on time of the switching circuits 510a, 510b, 510c, 510d is modulated by controller 503 to achieve the desired output parameters of a given DC pulse being generated and until the end of the pulse width of the DC pulse outputted by the discharge assembly 500 and applied to the target volume of soil.

As discussed in example embodiments above, control for output regulation is accomplished by setting a peak current value to be conducted through the soil and control feedback parameters (e.g., voltage and/or current) to achieve this peak current value. Controller 503 adjusts the pulse widths of the cycles of AC energy outputted from inverter circuitry 509 to provide conduction of peak current through the ground between the electrodes to manage the pests within the ground at the treatment location according to the described embodiment. Controller 503 may regulate the pulses using current control or voltage control to provide the peak current during treatment as described in some of the embodiments.

Referring to FIGS. 6A and 6B, an example conventional capacitive decay pulse waveform or energy profile 650 and an example square waveform or energy profile 660 of pulses of electrical energy outputted from the discharge assembly 500 and applied to a target volume of soil are shown, respectively. The use of a square waveform 660 such as shown in FIG. 6B increases the efficacy of the treatment compared with the use of the capacitive waveform 650 of FIG. 6A in some implementations as discussed further below.

The use of a capacitive decay pulse energy waveform is one example way to create an effective energy profile to control soil pests. An example method of generating a capacitive decay pulse uses self-commutating or passive SCR gates to initiate a capacitive decay discharge resulting in the capacitive decay waveform. In a capacitive decay discharge, the leading edge of the discharge or pulse has the greatest concentration of energy, where the applied energy is above a desired threshold voltage threshold (Vg) 652, such as 6 kV in one example, at the target volume of soil at the treatment location and is greater than or equal to the voltage threshold applied to deliver the desired voltage gradient. However, the energy delivered below Vg 652 (i.e., the trailing ˜95% of the stored energy) does not contribute to efficacy of treatment but is emptied from the charging capacitors before the SCR gates isolate and allow the capacitors to recharge. As the discharge occurs, the power dissipates precipitously as soon as the pulse is released, therefore the trailing ˜95% does not contribute to the treatment's efficacy. The delay created by the capacitive decay, as well as the time it takes to recharge the capacitors from emptied conditions, limits the frequency range that can be achieved, and ultimately the broad plant health potential resulting from the disclosed treatment methods.

In some embodiments described herein, it is desired to apply electrical pulses of increased frequency and variable duration or pulse width (e.g., using square wave pulses of FIG. 6B) to achieve increased plant health benefits compared with the pulses of FIG. 6A. The method of capacitive decay discharge also limits the ability to effectively target relatively small organisms, such as fungal pathogens, in large area applications where very high frequencies and very short pulses are desired to be utilized for treatment.

As discussed with respect to the example embodiment of the discharge assembly 500 of FIG. 5A-5F, digital switches (e.g., switching circuits 510a, 510b, 510c, 510d) are used as dual half bridge circuits to produce square waveforms shown in FIG. 6B with a <20% ripple in one disclosed embodiment. The square waveform energy content that provides efficacy of treatment is the portion of the applied energy that has a voltage equal to or above the voltage threshold Vg to deliver a predetermined energy gradient to the treatment target. In the depicted pulse examples, each pulse 650 of the capacitive decay waveform of FIG. 6A delivers approximately 0.8 Joules of effective energy (i.e., energy above Vg 652) while each pulse of the square waveform 660 of FIG. 6B delivers approximately 5 Joules of effective energy (i.e., energy above Vg 652 except for the risetime of the square waveform pulse is below Vg). With a high-fidelity square wave output of FIG. 6B (in comparison with the capacitive decay pulse of FIG. 6A) the amount of energy delivered that is below Vg 652 is greatly reduced.

The pulses of FIG. 6B encapsulate the peak voltage and current of the initial discharge of capacitively stored energy and output the most effective portion of the stored energy in every pulse of the square waveform in one embodiment. The digital switches of the embodiment of FIGS. 5A-5F are controlled to be closed to initiate the discharge of energy, then opened (commutated) to interrupt the discharge event and shut off the flow of the remaining stored energy from the capacitors (prior to emptying of the capacitors) resulting in quicker recharge of the capacitors and overall faster pulse discharge cycles than the use of a capacitive decay waveform of FIG. 6A. The use of square wave pulses enables delivery of peak energy density at a far wider range of frequencies to achieve desired plant health benefits.

In some embodiments, the discharge assembly is moved across the ground at the treatment location during the application of the pulses of electrical energy to the ground and the speed of the movement of discharge assembly may be used to determine a frequency of the application of the electrical pulses to the ground.

Speed of travel of the treatment apparatus during the application of electrical energy for treatment may be varied in a range of 1-20 feet per second in example embodiments. In one embodiment, the speed of travel is determined by the number of pulses the discharge assembly generates per second that are used to achieve a desired outcome with respect to a given target volume of soil. In one example mentioned above, the frequency of the pulses generated by the discharge assembly and applied for treatment to the soil may range from 1 to 10,000 pulses per second and speed of travel of the treatment apparatus is determined by how many pulses are to be stacked or applied to the target volume of soil at the treatment location in which the electrodes are engaged. For example, too slow a speed can result in arcing as the pulses are applied to the same volume of material and the current and voltage carrying capabilities of the volume break down. Conversely, if the treatment apparatus and discharge assembly traverse the treatment location at too fast a speed, areas of the treatment location may remain untreated if the pulse rate is not high enough as the electrodes may move through a portion of the volume of the treatment location without a pulse being discharged.

In one embodiment, frequency of pulses outputted by the discharge assembly and applied to the ground is fixed to a specific set value and the operator controls the speed of the treatment system applying the treatment to provide a desired number of pulses to a target volume of soil at the treatment location. The frequency of the pulses may also be dependent on the width and/or volume of soil at the treatment location. For example, at a 48″ treatment width, for nematodes on turf, and 400 Hz pulse rate, the speed of the treatment system does not exceed 2 mph. The speed of the treatment system and frequency of outputted pulses may vary from application to application.

An example method of treating organisms at a treatment location is discussed below according to one embodiment. Initially, a target organism to be treated or managed at a treatment location is identified. Thereafter, desired values of pulse parameters of applied electrical energy are identified to treat, control, manage or kill the target organism and the discharge assembly generates and applies the electrical energy in the form of a plurality of pulses to the treatment location containing the organisms.

In one embodiment, parameters of the applied pulses of electrical energy are empirically determined through experimentation. In one example, a voltage gradient of 40V/mm and total energy content delivered of 0.5 J/cc are used to treat endoparasitic nematodes. It is desirable in some embodiments to use increased pulse frequencies since efficacy increases as some target organisms are exposed to an increased number of pulses over a given period. Some considerations that are taken into account when determining the parameters for the applied electrical energy include the physical size of the organism, the organism's cellular characteristics and neurology, the conductive properties of the organism's environment (i.e., treatment location), electrical characteristics of the host crop (e.g., root matter, etc.) and/or average power available to be discharged from the discharge assembly.

In more specific examples, voltage is a selected value based on the physical size, anatomical characteristics (e.g., whether the target organism is an animal), cell size and structure of the target organism. Pulse width is determined by the characteristics of the load including the treatment location and the physical location of the pest or pathogen (e.g., endoparasitic: in the plant, or ectoparasitic: in the soil). Pulse width may also be used to determine a maximum frequency of the pulses of the applied electrical energy based upon the maximum average power available to be discharged from the discharge assembly. Frequency is determined in some embodiments by the physiological characteristics of the target organism which are susceptible to an electrical energy having a specific frequency or set of frequencies. Frequency of the pulses can also be determined by selecting the highest frequency that allows for maximum average power output from the discharge assembly in some embodiments.

In one example, average power is defined as:

$$Avg Power = Voltage * current * pulse width * frequency \qquad Equation 1$$

Example parameters of the applied electrical energy that may be used to control various organisms are shown in Table A wherein the treatments are provided at an approximately 40 kW average power output and with a given electrode spacing of approximately 20 cm from one another. The listed currents for the different pests to be managed may be referred to as the peak currents for the different treatments.

TABLE A

| Target | Voltage | Current | Pulse width | Frequency in Hz |
|---|---|---|---|---|
| Earthworms | 15 kV | 25 A | 5 ms | 21 |
| Phylloxera | 10 kV | 12 A | 1 ms | 333 |
| Endoparasitic | 10 kV | 22 A | .6 ms | 303 |
| Nematodes | | | | |
| Ectoparasitic | 20 kV | 30 A | .6 ms | 111 |
| Nematodes | | | | |
| Fungal | 40 kV | 30 A | .1 ms | 333 |
| Pathogen | | | | |

Additional details regarding treatment of soil pests and electrical parameters used during treatment are discussed in Ekaterini Riga, Jason D Crisp, Gordon J McComb, Jerry E Weiland, and Inga A Zasada; “Directed Energy System Technology For The Control of Soilborne Fungal Pathogens and Plant-Parasitic Nematodes;” Pest Management Science; Volume 76, Issue 6; Jan. 13, 2020; p. 2072-2078, the teachings of which are incorporated herein by reference.

Referring to FIG. 7, a plurality of waveforms are shown for various operations of the discharge assembly during treatment operations. Waveform 700 corresponds to a state of operation of the inverter circuitry 509 between an off (0) state where the inverter circuitry does not operate and on state (1) where the inverter circuitry outputs cycles of AC electrical energy.

Waveforms 702, 704 are used in embodiments that utilize synchronous bi-polar pulses where the waveform 702 includes positive voltage bi-polar pulses that are applied to one or more of the positive electrodes of the discharge assembly 500 and waveform 704 includes negative voltage pulses that are applied to one or more of the negative electrodes of the discharge assembly 500. The waveforms 702, 704 are synchronized with respect to time and are combined during application to the ground to generate a bi-polar square waveform 710 outputted by electrodes 520, 521 and as seen by the load (i.e., ground of the treatment location).

In the synchronized bi-polar output waveform 710, the output timing of the inverter devices (i.e., switching circuits 510a, 510b, 510c, 510d) is such that the primaries of the transformer circuitry 511 are driven with both dual half-bridge sections of the inverter circuitry 509 simultaneously into the dual output secondaries of transformer circuitry 511. The resulting output is a synchronized bi-polar output pulse and waveform 710 includes a plurality of synchronized bi-polar DC pulses formed by the combination of the positive and negative bi-polar voltage pulses of waveforms 702, 704. As mentioned above, the bi-polar pulses of waveforms 702, 704 may be used by applying the synchronized positive and negative pulses to respective electrodes for discharge to the treatment location.

Waveforms 706, 708 are used in embodiments that utilize asynchronous bi-polar pulses where the waveform 706 includes positive bi-polar voltage pulses that are applied to one or more of the positive electrodes of the discharge assembly 500 and waveform 708 includes negative bi-polar voltage pulses that are applied to one or more of the negative electrodes of the discharge assembly 500. The waveforms 706, 708 are not synchronized with respect to time and are combined to generate a bi-phasic bi-polar waveform 712 outputted by electrodes 520, 521 and as seen by the load (i.e., ground of the treatment location). In the bi-phasic bi-polar output waveform 712, the output timing of the inverter devices (i.e., switching circuits 510a, 510b, 510c, 510d) is such that one half of the dual bridge (e.g., switching circuits 510a, 510b) are driven for a predetermined output pulse width desired and then is shut off while simultaneously the other half of the dual bridge inverter (e.g., switching circuits 510c, 510d) is driven for a predetermined output pulse width. The resulting output waveform 712 includes a positive pulse of a predetermined pulse width followed by a pulse of a negative pulse of a predetermined pulse width for each on/off cycle of the inverter circuitry 509. Bi-phasic bi-polar waveform 712 includes a plurality of bi-phasic pulses formed by the combination of the positive and negative voltage pulses of waveforms 706, 708.

The positive voltage pulses of waveform 702 and the negative voltage pulses of waveform 704 are referenced to one another and the positive voltage pulses of waveform 706 and the negative voltage pulses of waveform 708 are each referenced to common. The combination of waveforms 706, 708 providing asynchronous bi-phasic and bi-polar pulses provides twice the output frequency for the same number of on/off cycles of the inverter circuitry which subjects the treatment location, and the pests which reside there, to a more constant flow of energy and reduced heat buildup in the switching circuits compared to the use of synchronous bi-polar pulses.

With a synchronous bi-polar pulse, the phase charge and the pulse charge are the same and always greater than zero. With a bi-phasic current (e.g., asynchronous bi-polar, bi-phasic pulse), the pulse charge is equal to the sum of the phase charges and can be viewed as two monophasic pulses of opposite directions, out of phase.

As mentioned above, some embodiments of the discharge assembly utilize a circuit architecture that generates a bi-polar (positive and negative) output pulse. A positive voltage pulse is applied to one electrode and a negative voltage pulse is applied to another electrode to form one of the square waveform pulses shown in FIG. 3 and waveform 710 of FIG. 7. If a bi-polar pulse including a positive pulse of +2 KV and a negative pulse of −2 KV are synchronized and applied to respective electrodes for application to the treatment location, then the pulse voltage across the electrodes is 4 KV. This output configuration using bi-polar pulses allows the use of one half the rated maximum voltage value of all components in the output assemblies providing a cost savings in materials, weight savings in assemblies, increased safety factors in fixed dimension spaces, and the use of less exotic materials.

Referring to FIGS. 8A and 8B, the underside and top side of an electrode assembly 800 are shown according to one example embodiment utilized to generate bi-phasic pulses. Synchronized bi-polar pulses may be generated using the electrode assembly 410 including sub-assemblies 411, 413 shown in FIGS. 1 and 2.

The electrode assembly 800 includes a single isolation member 802 that includes a plurality of recesses 804a, 804b, 804c that are configured to receive and electrically isolate a positively-biased buss bar 806a, a common bus bar 806b and a negatively-biased bus bar 806c. Positive buss bar 806a is coupled with positive output 520, common buss bar 806b is coupled with common node 522 and negative buss bar 806c is coupled with negative output 521 (positive and negative outputs 520, 521 and common node 522 are shown in FIGS. 5C and 5D).

Referring to 8A, the electrode assembly 800 provides respective electrodes 810a-810i at different locations that are aligned in a straight line that is substantially perpendicular to a direction of travel 602 of the treatment system along a swath of the treatment location according to one embodiment.

In one embodiment, electrodes 810a, 810e, 810i are biased at a positive voltage bias, electrodes 810b, 810d, 810f, 810h are at a common voltage bias and electrodes 810c, 810g are biased at a negative voltage bias. The positive electrodes 810a, 810e, 810i and negative electrodes 810c, 810g form a plurality of respective voltage gradients with adjacent common electrodes 810b, 810d, 810f, 810h across the swath of the treatment location 601. The conduction pathways of bi-phasic pulses are delivered from the positive electrodes to the common electrodes and from the common electrodes to the negative electrodes in one embodiment. A current emitted from a given electrode (e.g., positive electrode) may be applied to one or more electrodes (e.g., common electrodes) that are adjacent to the emitting electrode in one embodiment.

Movement of the treatment system moves the voltage gradients formed by pairs of electrodes across the treatment location during the traversal of the treatment location by the treatment system.

A plurality of electrically conductive fasteners 820 (e.g., steel bolts) are used to electrically connect electrodes 810a, 810e, 810i with positive buss bar 806a via orifices 807a, electrodes 810b, 810d, 810f, 810h with common buss bar 806b via orifices 807b, and electrodes 810c, 810g with negative buss bar 806c via orifices 807c. Electrically insulative fasteners (not shown) may additionally be utilized to fasten the electrodes with the buss bars for additional structural integrity in one embodiment.

Additional details regarding electrode assemblies and example electrode assemblies that may be utilized to generate and apply bi-polar pulses to a treatment location are discussed in co-pending PCT patent application PCT/US2022/037154, incorporated by reference above.

Some of the discharge assemblies discussed herein pulse the DC energy from a plurality of capacitors to create very short, abrupt surges of voltage and current. As mentioned above, the DC pulses applied to the treatment location may be generated and controlled by a plurality of digital switching devices which close and open to start and stop the flow of energy from the capacitors to the electrodes of the delivery apparatus engaged with a treatment location.

Some aspects of the disclosure described herein apply electrical energy to the treatment location in a plurality of DC pulses that are generated using capacitively stored electrical energy without complete discharge of the storage capacitors enabling increased rates of recharge of the capacitors and enabling pulses to be delivered to the treatment location at increased frequencies compared to some conventional arrangements where the capacitors are completely discharged. Some additional aspects of the disclosure enable delivery of an increased amount of electrical energy above a treatment voltage threshold to provide a desired voltage gradient to the treatment location to provide increased efficacy of treatment compared with some conventional methods.

According to some aspects of the disclosure, one or more parameters of the electrical energy being applied to the treatment location (e.g., voltage, current, pulse width, frequency) may be monitored and/or adjusted based upon varied soil conditions at the treatment locations to maintain the applied pulses of electrical energy within a desired range during treatment.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended aspects appropriately interpreted in accordance with the doctrine of equivalents.

Further, aspects herein have been presented for guidance in construction and/or operation of illustrative embodiments of the disclosure. Applicant(s) hereof consider these described illustrative embodiments to also include, disclose and describe further inventive aspects in addition to those explicitly disclosed. For example, the additional inventive aspects may include less, more and/or alternative features than those described in the illustrative embodiments. In more specific examples, Applicants consider the disclosure to include, disclose and describe methods which include less, more and/or alternative steps than those methods explicitly disclosed as well as apparatus which includes less, more and/or alternative structure than the explicitly disclosed structure.

1. A treatment system comprising:
a discharge assembly coupled with and configured to receive electrical energy from an input power source and to generate a plurality of pulses of electrical energy;
a plurality of electrodes coupled with the discharge assembly, and wherein the electrodes are configured to apply the pulses of electrical energy to material of a treatment location to manage a pest or pathogen within the material of the treatment location; and
wherein the pulses of electrical energy that are applied to the material of the treatment location are a plurality of square waveform pulses.

2. The system of claim 1 wherein the application of an individual one of the pulses of electrical energy to the material of the treatment location results in conduction of an electrical current through the material of the treatment location that is between the electrodes.

3. The system of claim 2 wherein the electrodes are configured to emit and receive the electrical current at different locations in the material of the treatment location and below a surface of the material of the treatment location.

4. The system of claim 1 wherein the discharge assembly comprises:
energy storage circuitry configured to store the electrical energy received from the input power source as direct current electrical energy;
inverter circuitry configured to convert the direct current electrical energy into alternating current electrical energy;
transformer circuitry configured to convert a parameter of the alternating current electrical energy providing converted electrical energy; and
rectifier circuitry configured to rectify the converted electrical energy into the pulses of electrical energy that are applied to the material of the treatment location.

5. The system of claim 1 wherein the discharge assembly is configured to generate a plurality of pulses of alternating current electrical energy corresponding to the square waveform pulses.

6. The system of claim 5 wherein the controller is configured to control pulse widths of the pulses of the alternating current electrical energy to control pulse widths of the pulses of electrical energy that are applied to the material of the treatment location.

7. The system of claim 1 wherein the pulses of electrical energy each have a voltage greater than a voltage threshold to manage the pest or pathogen.

8. The system of claim 7 wherein an individual one of the pulses of electrical energy has a voltage greater than the voltage threshold for substantially an entirety of the pulse width of the individual pulse of electrical energy.

9. The system of claim 7 wherein the discharge assembly comprises energy storage circuitry configured to store the electrical energy received from the input power source at a voltage greater than the voltage threshold.

10. The system of claim 9 wherein a voltage of the energy storage circuitry remains at least substantially equal to or greater than the voltage threshold during the generation of the pulses and the application of the pulses to the material of the treatment location.

11. The system of claim 1 wherein the discharge assembly comprises a controller configured to control a pulse width of each of the pulses of electrical energy.

12. The system of claim 11 wherein the discharge assembly comprises energy storage circuitry configured to store the electrical energy received from the input power source, each of the pulses of electrical energy has a voltage greater than a voltage threshold to manage the pest or pathogen, and the controller controls is configured to control the pulse width of each of the pulses of electrical energy to maintain a voltage of the energy storage circuitry at least substantially equal to or greater than the voltage threshold during the application of the pulses of electrical energy to the material.

13. The system of claim 1 further comprising storage circuitry configured to store a plurality of different values of a parameter of the pulses of electrical energy for managing different types of the pest or pathogen, and a controller configured to select one of the values of the parameter to control the generation of the pulses of electrical energy to manage the pest or pathogen present within the material of the treatment location.

14. The system of claim 13 wherein the parameter includes at least one of voltage, current, frequency and pulse width of the pulses of electrical energy.

15. The system of claim 1 wherein the pulses of electrical energy each have a voltage in a range of 10 VDC to 100 kVDC.

16. The system of claim 1 wherein each of the square waveform pulses has a risetime of about 10 microseconds or less.

17. The system of claim 1 wherein the pulses are applied to the material of the treatment location at a frequency that provides an at least substantially maximum average power output from the discharge assembly to the material of the treatment location.

18. (canceled)

19. The system of claim 1 wherein the discharge assembly is configured to generate the square waveform pulses at a frequency corresponding to at least a substantially maximum average power output from the discharge assembly.

20. The system of claim 1 wherein the discharge assembly comprises a switching circuit and a controller configured to control selective opening and closing of the switching circuit at a plurality of moments in time to generate the square waveform pulses.

21. The system of claim 20 wherein the discharge assembly comprises an inverter circuitry that includes the switching circuit.

22. The system of claim 1 wherein the discharge assembly comprises a controller configured to monitor at least one of the pulses of electrical energy that is applied to the material of the treatment location and to adjust a parameter of another of the pulses of electrical energy that is applied to the material of the treatment location as a resulting of the monitoring.

23. The system of claim 1 wherein the discharge assembly comprises inverter circuitry configured to output pulses of alternating current electrical energy to generate the square waveform pulses, and a controller is configured to adjust pulse widths of the pulses of alternating current electrical energy to adjust a parameter of the square waveform pulses.

24. The system of claim 23 wherein the controller is configured to adjust the parameter of the pulses to conduct peak current through the material of the treatment location.

25. The system of claim 1 wherein the discharge assembly is configured to output a positive voltage pulse to one of the electrodes and a negative voltage pulse to another of the electrodes to generate one of the square waveform pulses.

26. The system of claim 25 wherein the discharge assembly is configured to output the positive and negative voltage pulses synchronized with respect to time to generate the one square waveform pulse comprising a bi-polar pulse.

27. The system of claim 25 wherein the discharge assembly is configured to output the positive and negative voltage pulses not synchronized with respect to time to generate the one square waveform pulse comprising a bi-phasic pulse.

28. The system of claim 1 further comprising a user interface configured to receive an input from a user, and wherein the discharge assembly is configured to use one of a plurality of values of a parameter of the pulses of electrical energy to generate the pulses of electrical energy as a result of the receiving the input.

29. The system of claim 1 wherein the treatment system is configured to be moved across the treatment location during the application of the pulses of electrical energy to the material of the treatment location, and wherein a controller is configured to use a speed of the treatment system to determine a frequency of the application of the pulses of electrical energy to the material of the treatment location.

30-69. (canceled)

70. The system of claim 1 wherein each of the pulses of electrical energy has a current in a range of 1 to 10,000 Amps through the material of the treatment location.

71. The system of claim 1 wherein the pulses of electrical energy are applied to the material of the treatment location at a frequency of 1 Hz to 10 KHz.

72. The system of claim 1 wherein the treatment system is configured to be moved across the treatment location during the application of the pulses of electrical energy to the material of the treatment location and the application of the pulses of electrical energy to the material of the treatment location generates a voltage gradient between the electrodes that is moved through different volumes of the material of the treatment location during the movement of the treatment system.

73. The system of claim 72 wherein the voltage gradient is continuously generated during the movement of the treatment system.

74. The system of claim 1 wherein the application of the pulses of electrical energy to the material of the treatment location generates a voltage gradient of 20 V/mm or greater across a volume of the material of the treatment location that is between the electrodes.

75. The system of claim 74 wherein the application of the pulses of electrical energy to the material of the treatment location generates the voltage gradient of 200 V/mm or less across a volume of the material of the treatment location that is between the electrodes.

76. The system of claim 1 wherein the electrodes are configured to contact the material of the treatment location during the application of the pulses electrical energy to the material of the treatment location.

77. The system of claim 1 wherein the material is soil.

78. The system of claim 1 wherein the application of the pulses of electrical energy to the material of the treatment location effects an in-situ management of the pest or pathogen within the material of the treatment location.

79. The system of claim 1 wherein the material of the treatment location includes plant matter.
